(19) United States
(12) Patent Application Publication
(10) Pub. No.: US 2024/0331829 A1
(43) Pub. Date: Oct. 3, 2024

(54) QUANTITATIVE SYSTEMS PHARMACOLOGY IN CYTOKINE-BASED CANCER IMMUNOTHERAPY

(71) Applicant: Genentech, Inc., South San Francisco, CA (US)

(72) Inventors: Mohammad Jafarnejad, San Francisco, CA (US); Louis Russell Joslyn, San Francisco, CA (US); Iraj Hosseini, San Francisco, CA (US); Saroja Ramanujan, San Francisco, CA (US)

(21) Appl. No.: 18/626,741

(22) Filed: Apr. 4, 2024

Related U.S. Application Data
(63) Continuation of application No. PCT/US2022/077614, filed on Oct. 5, 2022.
(60) Provisional application No. 63/252,483, filed on Oct. 5, 2021.

(51) Int. Cl. G16H 20/17; G16B 15/30

(52) U.S. Cl. CPC G16H 20/17; G16B 15/30

(57) ABSTRACT

The present disclosure relates generally to the field of immunology and oncology, and particularly to systems and methods for use in treating various disorders, such as those associated with cell signaling mediated by cytokines. Some embodiments of the disclosure provide quantitative system pharmacology (QSP) models developed to capture the complex interactions between drug pharmacokinetics and the differential expansion of multiple immune cell subsets across tissues and tumor.

### CROSS-REFERENCE TO RELATED APPLICATIONS

This application claims the benefit of priority to U.S. Provisional Patent Application Ser. No. 63/252,483, filed on Oct. 5, 2021. The disclosure of the above-referenced application is herein expressly incorporated by reference it its entirety, including any drawings.

### FIELD

The present disclosure relates generally to the field of immunology and medicine, including systems and methods useful for cytokine-based immunotherapy.

### BACKGROUND

Cytokine-based immunotherapy is a promising field in cancer treatment, since cytokines, as proteins of the immune system, are able to modulate the host immune response toward cancer cells, as well as directly induce tumor cell death. This is because these secreted proteins play important roles for cell signaling, cell-cell communication, and the modulation of proliferation and differentiation of specific cells that express the relevant receptors. Several cytokine-based immunotherapies have been demonstrated to be effective in the treatment of several disorders, including many types of cancers.

For example, in the past decades, discovery of the activity of pro-inflammatory cytokines in cancer led to several clinical trials that showed mild activity in patients treated with interferon-alpha (IFN-α), interleukin-2 (IL-2) and interleukin-15 (IL-15). However, the clinical activity of these replacement cytokine therapies has been limited due to a variety of issues, including the short half-life of the molecules and a small therapeutic window mainly because of safety issues. In addition, since a low dose monotherapy with some cytokines has no significant therapeutic results and a high dose treatment leads to a number of side effects caused by the pleiotropic effect of cytokines, the problem of understanding the influence of cytokines on the immune cells involved in the pro- and anti-tumor immune response remains a pressing one.

Thus, there exists a need for new approaches to efficiently support and optimize the safety and effectiveness of cytokine-based immunotherapies.

Recent advances in the field of pharmacometrics suggest that pharmacometric analyses can potentially improve the efficiency of the drug development process by simulating complex and diverse data drug information for populations or clinical scenarios that are difficult to test for practical or ethical reasons. Pharmacometrics generally involves using quantitative analysis and modelling and simulation (M&S) approaches to inform and enhance drug development and regulatory review. Pharmacometrics also encompasses quantitative system pharmacology (QSP) and model-informed drug development (MIDD) approaches. Common applications of pharmacometric analyses for regulatory drug submissions include using modeling and simulation to: (1) aid in the design of clinical trials, (2) identify clinically relevant covariates, (3) characterize pharmacokinetic and exposure-response profiles, (4) molecule design, 5) preclinical to clinical translation, and (6) extrapolate efficacy to special populations, for example, pediatrics or geriatrics. Using pharmacometrics can provide efficient and cost-effective approaches to support and optimize drug safety and effectiveness.

In addition, pharmacometric approaches play an increasing role in developing drug submissions and clinical trial applications (CTAs). Regulators are working to find the best way to evaluate pharmacometric approaches for drug evaluation and regulatory decision-making.

The present disclosure relates generally to, inter alia, methods and systems of modeling the pharmacodynamics and pharmacokinetics of cytokine-based therapies.

### SUMMARY

The present disclosure relates generally to the fields of immunology and oncology, and particularly to systems and methods for use in treating various disorders, such as those associated with cell signaling mediated by cytokines.

Methods, systems, and articles of manufacture, including computer program products, are provided for quantitative systems pharmacology in cytokine-based immunotherapy. In one aspect, a method includes determining, by at least one data processor and after delivery of a cytokine molecule, (a) a dynamics parameter corresponding to a plurality of target lymphocytes within a central compartment of a patient and (b) a binding parameter between the cytokine molecule and the plurality of target lymphocytes within the central compartment. The plurality of target lymphocytes include a CD8+ T cell, a CD4+ T cell, a CD4−/CD8− (Double Negative) T cell, and a natural killer (NK) cell. The method may include determining, by the at least one data processor, (a) a first target cell trafficking rate of the plurality of target lymphocytes between the central compartment and a tight compartment of the patient and (b) a first cytokine partitioning rate of the cytokine molecule between the central compartment and the tight compartment. The method may include determining, by the at least one data processor, (a) the dynamics parameter corresponding to the plurality of target lymphocytes within the tight compartment and (b) the binding parameter between the cytokine molecule and the plurality of target lymphocytes within the tight compartment. The method may include determining, by the at least one data processor, (a) a second target cell trafficking rate of the plurality of target lymphocytes between the central compartment and a leaky compartment of the patient and (b) a second cytokine partitioning rate of the cytokine molecule between the central compartment and the leaky compartment. The method may include determining, by the at least one data processor, (a) the dynamics parameter corresponding to the plurality of target lymphocytes within the leaky compartment and (b) the binding parameter between the cytokine molecule and the plurality of target lymphocytes within the leaky compartment. The method may include determining, by the at least one data processor, (a) a third target cell trafficking rate of the plurality of target lymphocytes between the central compartment and a tumor compartment of the patient and (b) a third cytokine partitioning rate of the cytokine molecule between the central compartment and the tumor compartment. The method may include determining, by the at least one data processor, (a) the dynamics parameter corresponding to the plurality of target lymphocytes within the tumor compartment and (b) the binding parameter between the cytokine molecule and the plurality of target lymphocytes within the tumor compartment. The method may include determining, by the at least one data processor, a fourth cytokine partitioning rate of the cytokine molecule from each of the tight compartment, the leaky compartment, and the tumor compartment, to a lymph compartment of the patient. The method may include determining, by the at least one data processor and based on the dynamics parameter, the binding parameter, the first target cell trafficking rate, the second target cell trafficking rate, the third target cell trafficking rate, the first cytokine partitioning rate, the second cytokine partitioning rate, the third cytokine partitioning rate, and the fourth cytokine partitioning rate, a distribution of each of the plurality of target lymphocytes in the central compartment, the tight compartment, the leaky compartment, and the tumor compartment, over time.

In some variations, one or more of the features disclosed herein including the following features can optionally be included in any feasible combination. In some variations, the method includes determining, based at least on the distribution, a cancer immunotherapy for treating a tumor. The cancer immunotherapy includes a dose of the cytokine molecule and a dosing frequency for delivering the dose of the cytokine molecule.

In some variations, the distribution includes: a first distribution corresponding to the cytokine molecule delivered to the patient at a first time point and a second distribution corresponding to the cytokine molecule delivered to the patient at a second time point. The method includes: determining, by the at least one data processor and based at least on the first distribution and the second distribution, a response of a second patient to a cancer immunotherapy including the cytokine molecule.

In some variations, the method includes determining, based at least on the response of the second patient to the cancer immunotherapy, a treatment plan for the second patient.

In some variations, the method includes: administering the cancer immunotherapy according to the treatment plan.

In some variations, each of the plurality of target lymphocytes includes a first subpopulation associated with high PD1 expression and a second subpopulation associated with low PD1 expression. The method further includes: determining the dynamics parameter for the first subpopulation and the second subpopulation for each of the plurality of target lymphocytes in the central compartment, the tight compartment, the leaky compartment, and the tumor compartment.

In some variations, the dynamics parameter includes at least one of a quantity, a proliferation, an expansion, a contraction, a persistence, and a differentiation of the plurality of target lymphocytes within the central compartment, the tight compartment, the leaky compartment, and the tumor compartment.

In some variations, the binding parameter includes at least one of an affinity and an avidity.

In some variations, the binding parameter includes a concentration of free receptors at a surface of each of the plurality of target lymphocytes within the central compartment, the tight compartment, the leaky compartment, and the tumor compartment, a concentration of single bound drug: receptor complexes at the surface of each of the plurality of target lymphocytes within the central compartment, the tight compartment, the leaky compartment, and the tumor compartment, and a concentration fully bound cytokine molecule complexes at the surface of each of the plurality of target lymphocytes within the central compartment, the tight compartment, the leaky compartment, and the tumor compartment.

In some variations, the binding parameter is associated with expression of at least one of an interleukin 15 (IL-15) receptor and a PD1 receptor on a surface of each of the plurality of target lymphocytes within the central compartment, the tight compartment, the leaky compartment, and the tumor compartment.

In some variations, the method includes determining, by the at least one data processor, a fifth cytokine partitioning rate of the cytokine molecule from the lymph compartment to the central compartment.

In some variations, the cytokine molecule includes interleukin 2 (IL-2), interleukin 7 (IL-7), interleukin 12 (IL-12), interleukin 15 (IL-15), interleukin 21 (IL-21), or interferon.

In some variations, the cytokine molecule includes Xmab24306, PD1/IL15 TaCk, or RSV IL-15 TaCk.

In some variations, the cytokine molecule includes an engineered cytokine.

In some variations, the engineered cytokine agonizes its cognate receptor.

In some variations, the cytokine molecule stimulates an immune system of the patient.

In some variations, the cytokine molecule is a bispecific molecule including a first arm and a second arm. Interleukin 15 receptors (IL-15R) of each of the plurality of target lymphocytes bind to the first arm of the bispecific molecule. PD1 receptors of each of the plurality of target lymphocytes bind to the second arm of the bispecific molecule.

In some variations, the cytokine molecule is a multivalent polypeptide including a first polypeptide region capable of binding to a first target and a second polypeptide region capable of binding to a second target. The first polypeptide region is operably linked to the second polypeptide region.

In some variations, the first target is a first receptor of the cytokine molecule, and the second target is a second receptor of the cytokine molecule.

In some variations, the first target is an interleukin 15 receptor (IL-15R), and the second target is a PD1 receptor.

In some variations, a binding affinity of the second polypeptide region to the second target is increased via avidity when the first polypeptide region binds to the first target.

In some variations, a binding of the second polypeptide region to the second target has Kd of second interaction that is higher when the first polypeptide region binds to the first target.

In one aspect, there is provided a system. The system may include at least one processor and at least one memory. The at least one memory may store instructions that result in operations when executed by the at least one processor. The operations may include: determining, after delivery of a cytokine molecule, (a) a dynamics parameter corresponding to a plurality of target lymphocytes within a central compartment of a patient and (b) a binding parameter between the cytokine molecule and the plurality of target lymphocytes within the central compartment. The plurality of target lymphocytes include a CD8+ T cell, a CD4+ T cell, a CD4−/CD8− (Double Negative) T cell, and a natural killer (NK) cell. The operations may further include determining (a) a first target cell trafficking rate of the plurality of target lymphocytes between the central compartment and a tight compartment of the patient and (b) a first cytokine partitioning rate of the cytokine molecule between the central compartment and the tight compartment. The operations may further include determining (a) the dynamics parameter corresponding to the plurality of target lymphocytes within the tight compartment and (b) the binding parameter between the cytokine molecule and the plurality of target lymphocytes within the tight compartment. The operations may further include determining (a) a second target cell trafficking rate of the plurality of target lymphocytes between the central compartment and a leaky compartment of the patient and (b) a second cytokine partitioning rate of the cytokine molecule between the central compartment and the leaky compartment. The operations may further include determining (a) the dynamics parameter corresponding to the plurality of target lymphocytes within the leaky compartment and (b) the binding parameter between the cytokine molecule and the plurality of target lymphocytes within the leaky compartment. The operations may further include determining (a) a third target cell trafficking rate of the plurality of target lymphocytes between the central compartment and a tumor compartment of the patient and (b) a third cytokine partitioning rate of the cytokine molecule between the central compartment and the tumor compartment. The operations may further include determining (a) the dynamics parameter corresponding to the plurality of target lymphocytes within the tumor compartment and (b) the binding parameter between the cytokine molecule and the plurality of target lymphocytes within the tumor compartment. The operations may further include determining a fourth cytokine partitioning rate of the cytokine molecule from each of the tight compartment, the leaky compartment, and the tumor compartment, to a lymph compartment of the patient. The operations may further include determining, based on the dynamics parameter, the binding parameter, the first target cell trafficking rate, the second target cell trafficking rate, the third target cell trafficking rate, the first cytokine partitioning rate, the second cytokine partitioning rate, the third cytokine partitioning rate, and the fourth cytokine partitioning rate, a distribution of each of the plurality of target lymphocytes in the central compartment, the tight compartment, the leaky compartment, and the tumor compartment, over time.

In one aspect, there is provided a non-transitory computer-readable medium storing instructions, which when executed by at least one data processor, result in operations. The operations may include: determining, after delivery of a cytokine molecule, (a) a dynamics parameter corresponding to a plurality of target lymphocytes within a central compartment of a patient and (b) a binding parameter between the cytokine molecule and the plurality of target lymphocytes within the central compartment. The plurality of target lymphocytes include a CD8+ T cell, a CD4+ T cell, a CD4−/CD8− (Double Negative) T cell, and a natural killer (NK) cell. The operations may further include determining (a) a first target cell trafficking rate of the plurality of target lymphocytes between the central compartment and a tight compartment of the patient and (b) a first cytokine partitioning rate of the cytokine molecule between the central compartment and the tight compartment. The operations may further include determining (a) the dynamics parameter corresponding to the plurality of target lymphocytes within the tight compartment and (b) the binding parameter between the cytokine molecule and the plurality of target lymphocytes within the tight compartment. The operations may further include determining (a) a second target cell trafficking rate of the plurality of target lymphocytes between the central compartment and a leaky compartment of the patient and (b) a second cytokine partitioning rate of the cytokine molecule between the central compartment and the leaky compartment. The operations may further include determining (a) the dynamics parameter corresponding to the plurality of target lymphocytes within the leaky compartment and (b) the binding parameter between the cytokine molecule and the plurality of target lymphocytes within the leaky compartment. The operations may further include determining (a) a third target cell trafficking rate of the plurality of target lymphocytes between the central compartment and a tumor compartment of the patient and (b) a third cytokine partitioning rate of the cytokine molecule between the central compartment and the tumor compartment. The operations may further include determining (a) the dynamics parameter corresponding to the plurality of target lymphocytes within the tumor compartment and (b) the binding parameter between the cytokine molecule and the plurality of target lymphocytes within the tumor compartment. The operations may further include determining a fourth cytokine partitioning rate of the cytokine molecule from each of the tight compartment, the leaky compartment, and the tumor compartment, to a lymph compartment of the patient. The operations may further include determining, based on the dynamics parameter, the binding parameter, the first target cell trafficking rate, the second target cell trafficking rate, the third target cell trafficking rate, the first cytokine partitioning rate, the second cytokine partitioning rate, the third cytokine partitioning rate, and the fourth cytokine partitioning rate, a distribution of each of the plurality of target lymphocytes in the central compartment, the tight compartment, the leaky compartment, and the tumor compartment, over time.

In various aspects and embodiments of the disclosure provide QSP models developed to capture the complex interactions between drug pharmacokinetics (PK) and the differential expansion of multiple immune cell subsets across tissues and tumor. In particular, the experimental results described herein demonstrate that the QSP model can be used to support preclinical, translational and early clinical development of engineered polypeptide constructs, e.g., engineered cytokines, for cancer immunotherapy.

In one aspect, disclosed herein are systems that include (a) at least one data processor; and (b) at least one memory storing instructions, which when executed by at least one data processor, result in operations comprising: (i) determining a first parameter of a first binding affinity between a first region of a molecule of interest and a first target on an immune cell; (ii) determining a second parameter of a second binding affinity between a second region of the molecule of interest and a second target on the immune cell, wherein the first binding affinity and the second binding affinity are measured in multiple compartments; and (iii) generating, based at least on the first parameter and the second parameter, an output indicating a pharmacokinetic pharmacodynamic (PKPD) relationship for the molecule of interest.

Non-limiting exemplary embodiments of the systems of the disclosure can include one or more of the following features. In some embodiments, the molecule of interest is an agonist of a cytokine. In some embodiments, the cytokine is an immunostimulatory cytokine. In some embodiments, the cytokine is interleukin 2 (IL-2), interleukin IL-7 (IL-7), interleukin 12 (IL-12), interleukin 15 (IL-15), interleukin 21 (IL-21), or interferon.

In some embodiments, the molecule of interest is an engineered cytokine. In some embodiments, the engineered cytokine agonizes its cognate receptor. In some embodiments, the molecule of interest is a multivalent polypeptide including: (i) a first polypeptide region capable of binding a first target, and (ii) a second polypeptide region capable of binding to a second target, wherein the first polypeptide region is operably linked to the second polypeptide region. In some embodiments, the first polypeptide region is capable of binding to IL-15 receptor (CD122) or variant thereof. In some embodiments, the first target is IL-15R or a variant thereof. In some embodiments, the second polypeptide region is capable of binding to PD1 or a variant thereof. In some embodiments, the second target is PD1 or a variant thereof. In some embodiments, the molecule of interest is configured such that the binding avidity of the second region to the second target is increased when the first region binds to the first target. In some embodiments, the molecule of interest is configured such that the binding of the second region to the second target has K_{d} of second interaction that is higher when the first region binds to the first target. In some embodiments, the systems of the disclosure include modeling the mechanisms of drug pharmacokinetics (PK), pharmacodynamics (PD), dynamic target-mediated drug disposition (TMDD), cellular expansion, and target receptor expression in a plurality of immune cells, tissues, and/or compartments. In some embodiments, the systems include modeling binding avidity of the first region and/or the second region to the respective first target and second target. In some embodiments, the modeling of binding avidity comprises tracking one or more of free receptors, bound receptors, receptor: drug: receptor complex, and cells over time. In some embodiments, the plurality of compartments include one or more tight compartments, leaky compartments, tumor compartments, lymph compartments, central compartments, or a combination of any thereof. In some embodiments, the one or more tight compartments includes muscle, skin, adipose, and/or brain. In some embodiments, the one or more leaky compartments includes liver, kidney, heart, lung, spleen, live nude, bone, and/or intestine. In some embodiments, the plurality of immune cells includes CD8⁺ T cells, CD4⁺ T cells, gamma delta T cells, natural killer (NK) cells, or a combination of any thereof. In some embodiments, the CD8⁺ T cell, CD4⁺ T cell, gamma delta T cell are PD1^{hi} cells. In some embodiments, the NK cells are CD56^{hi} and PD1^{hi} cells. In some embodiments, the NK cells are PD1^{lo} cells and CD16⁺ cells. In some embodiments, the plurality of immune cells includes one or more subpopulations of cells that individually express the first target, the second target, or both targets.

In one aspect, provided herein are methods for preparing a quantitative pharmacological model for evaluating/predicting PKPD relationship following treatment with a molecule of interest in a subject. The methods include (i) determining a first parameter of a first binding affinity between a first region of a molecule of interest and a first target on an immune cell; (ii) determining a second parameter of a second binding affinity between a second region of the molecule of interest and a second target on the immune cell, wherein the first binding affinity and the second binding affinity are measured in multiple compartments; and (iii) generating, based at least on the first parameter and the second parameter, an output indicating a pharmacokinetic pharmacodynamic (PKPD) relationship for the molecule of interest.

In another aspect, provided herein are methods for evaluating a molecule of interest for cell proliferation capabilities, the methods include executing a system as disclosed herein.

In another aspect, provided herein are methods for evaluating lymphocyte expansion in different tissues and tumors, the methods include executing a system as disclosed herein.

Non-limiting exemplary embodiments of the methods of the disclosure can include one or more of the following features. In some embodiments, the molecule of interest is an agonist of a cytokine. In some embodiments, the cytokine is an immunostimulatory cytokine. IN some embodiments, the cytokine is interleukin 2 (IL-2), interleukin IL-7 (IL-7), interleukin 12 (IL-12), interleukin 15 (IL-15), interleukin 21 (IL-21), or interferon.

In some embodiments, the molecule of interest is an engineered cytokine. In some embodiments, the engineered cytokine agonizes its cognate receptor. In some embodiments, the molecule of interest is a multivalent polypeptide including: (i) a first polypeptide region capable of binding a first target, and (ii) a second polypeptide region capable of binding to a second target, wherein the first polypeptide region is operably linked to the second polypeptide region. In some embodiments, the first polypeptide region is capable of binding to IL-15 receptor (CD122) or variant thereof. In some embodiments, the first target is IL-15R or a variant thereof. In some embodiments, the second polypeptide region is capable of binding to PD1 or a variant thereof. In some embodiments, the second target is PD1 or a variant thereof. In some embodiments, the molecule of interest is configured such that the binding avidity of the second region to the second target is increased when the first region binds to the first target. In some embodiments, the molecule of interest is configured such that the binding of the second region to the second target has K_{d} of second interaction that is higher when the first region binds to the first target. In some embodiments, the methods of the disclosure include modeling the mechanisms of drug pharmacokinetics (PK), pharmacodynamics (PD), dynamic target-mediated drug disposition (TMDD), cellular expansion, and target receptor expression in a plurality of immune cells, tissues, and/or compartments. In some embodiments, the methods include modeling binding avidity of the first region and/or the second region to the respective first target and second target. In some embodiments, the modeling of binding avidity comprises tracking one or more of free receptors, bound receptors, receptor: drug: receptor complex, and cells over time. In some embodiments, the plurality of compartments include one or more tight compartments, leaky compartments, tumor compartments, lymph compartments, central compartments, or a combination of any thereof. In some embodiments, the one or more tight compartments includes muscle, skin, adipose, and/or brain. In some embodiments, the one or more leaky compartments includes liver, kidney, heart, lung, spleen, live nude, bone, and/or intestine.

In some embodiments, the plurality of immune cells includes CD8⁺ T cells, CD4⁺ T cells, gamma delta T cells (e.g., double negative T cell), natural killer (NK) cells, or a combination of any thereof. In some embodiments, the CD8⁺ T cell, CD4⁺ T cell, gamma delta T cell are PD1^{hi} cells. In some embodiments, the NK cells are CD56^{hi} and PD1^{hi} cells. In some embodiments, the NK cells are PD1^{lo} cells and CD16⁺ cells.

In some embodiments, the plurality of immune cells includes one or more subpopulations of cells that individually express the first target, the second target, or both targets.

The foregoing summary is illustrative only and is not intended to be in any way limiting. In addition to the illustrative embodiments and features described herein, further aspects, embodiments, objects and features of the disclosure will become fully apparent from the drawings and the detailed description and the claims.

### BRIEF DESCRIPTION OF THE DRAWINGS

FIG. 1 schematically illustrates the potential of IL-15 in immunotherapy.

FIG. 2 depicts two exemplary engineered cytokines that target the IL-15 receptor in accordance with some embodiments of the disclosure.

FIG. 3A schematically illustrates an exemplary QSP model, in accordance with some embodiments of the disclosure, which is developed to capture pharmacokinetics (PK) and pharmacodynamics (PD) for different molecules targeting IL-15 signaling.

FIG. 3B schematically illustrates an exemplary unit of interactions in a compartment of a QSP model, in accordance with some embodiments of the disclosure;

FIG. 4 illustrates an example workflow for calibrating and validating a QSP model, in accordance with some embodiments of the disclosure.

FIG. 5 schematically illustrates a calibrated model that captures PK and PD data from experiments performed with an engineered cytokine in cynomolgus monkeys.

FIG. 6 schematically illustrates a calibrated model that captures PK and PD data from experiments performed with an engineered cytokine in cynomolgus monkeys.

FIG. 7 provides a list of non-limiting exemplary questions that can be addressed by using the QSP framework described herein.

FIGS. 8A-8D illustrate cell-fold change in cell populations following molar-matched doses of Xmab24306 or PD1 IL-15 TaCk, in accordance with some embodiments of the disclosure.

FIG. 9 schematically summarizes the results of experiments illustrating that virtual cohort simulations capture dynamics and variability of cytokine molecule data in cynomolgus monkeys.

FIG. 10 depicts different scenarios for translation from monkey to human.

FIG. 11A schematically summarizes the results of experiments comparing CD8 expansion for different clinical scenarios for an engineered cytokine molecule.

FIG. 11B schematically summarizes the results of experiments comparing CD8 expansion for different clinical scenarios for an engineered cytokine molecule.

FIG. 12 illustrates cellular response and bound drug in the central and leaky compartments following treatment, in accordance with some embodiments of the disclosure.

FIG. 13 illustrates that virtual Cohort captures dynamics and variability of preclinical, multi-dose PD1/IL-15 TaCk PKPD dataset in cynomolgus macaques, in accordance with some example embodiments of the disclosure.

FIG. 14 depicts a system diagram illustrating an example of an analysis system, in accordance with some example embodiments.

FIG. 15 depicts a flowchart illustrating an example of a process for determining a distribution of target lymphocytes, in accordance with some example embodiments.

FIG. 16 depicts a flowchart illustrating an example of a process for QSP modeling, in accordance with some example embodiments.

FIG. 17 depicts a block diagram illustrating a computing system, in accordance with some example embodiments.

### DETAILED DESCRIPTION OF THE DISCLOSURE

The present disclosure relates generally to the field of immunology and oncology, and particularly to systems and methods for use in treating various disorders, such as those associated with cell signaling mediated by cytokines. As described in greater detail below, some embodiments of the disclosure provide a QSP model to capture the complex interactions between drug pharmacokinetics (PK) and the differential expansion of multiple immune cell subsets across tissues and tumor. In particular, the experimental results described herein demonstrate that the QSP model can be used to support preclinical, translational and early clinical development of engineered cytokines for cancer immunotherapy, as exemplified by multivalent polypeptides engineered to target cell signaling mediated by interleukin 15 (IL-15) (see, e.g., Example 1).

Cytokines are secreted proteins that play important roles for cell signaling, and cell-cell communication, and the modulation of proliferation and differentiation of specific cells that express the relevant receptors. For example, cytokines are crucial mediators of the immune response against infection or disease. These secreted proteins play important roles in cell signaling, proliferation, death, or cell to cell communication. As monotherapies or in combination with other anticancer immunotherapies, several cytokine-based immunotherapies have demonstrated efficacy in the treatment of cancer but can suffer from poor drug exposure. Discovery of the activity of pro-inflammatory cytokines in cancer led to several clinical trials that showed mild activity in patients treated with interferon-alpha (IFN-α), interleukin-2 (IL-2) and interleukin-15 (IL-15). The clinical activity of these replacement cytokine therapies was, however, limited due to the short half-life of the molecules and a small therapeutic window mainly because of safety issues. In the past decade, novel molecular formats have been used to address these limitations by engineering cytokines with increased half-life and/or targeting arms.

In particular, since lymphocyte-targeting IL-15 molecules have shown potential to alter the tumor-immune environment by expanding effector cell populations, a QSP model, consistent with embodiments of the current subject matter, captures the complex interactions between drug pharmacokinetics (PK) and the differential expansion of multiple immune cell subsets across tissues and tumor sites. The QSP model described herein supports preclinical, translational and early clinical development of IL-15 molecules, and can be extended to support other engineered cytokines for cancer immunotherapy.

IL-15 primarily induces proliferation of NK cells and T-cells as a function of their IL-15RA (CD215), IL-15RB (CD122) and IL-2RG (CD132) expression levels. A fused IL-15/IL-15RA on half-life extended Fc domain (named XmAb24306) was developed with reduced potency to improve tolerability of the molecule. The molecule is currently being tested in the clinic as a single agent or in combination with atezolizumab (anti-PDL1) in patients with locally advanced or metastatic solid tumors. A PD1-targeted candidate (PD1/IL-15 TaCk) with a similar IL-15 targeting arm was also developed to promote selective expansion of specific T-cell populations. Consistent with embodiments of the current subject matter, the QSP model described herein illustrates the pharmacokinetics (PK) of PD1/IL-15 TaCk, among other cytokine-based molecules, due to target-mediated drug disposition (TMDD) and characterizes the resulting cell expansion that could differentiate between cytokine-based molecules, such as PD1/IL-15 TaCk and XmAb24306.

Generally, natural killer (NK) cells and T cells share certain phenotypic and functional similarities and both can respond rapidly in an antitumor response, enabling increased anti-tumor cytotoxicity. Consequently, a high-level objective of cancer immunotherapies is to modulate the magnitude of each of these cell populations. As a relatively new cytokine-based immunotherapy, engineered IL-15 molecules appear to effectively induce the activation and proliferation of NK and T cells across multiple different formats and administrations. However, as noted herein, the translational potential of these molecules is generally difficult to predict due to: 1) a time-dependent PK/PD relationship that depends on the extent of target cell expansion, 2) unknown competition between target cell populations that could potentially bind to the drug, and 3) unknown relationships between tissue and blood which could limit the utility of blood sampling as an indicator of events in the tissues.

As described in greater detail below, the QSP model consistent with embodiments of the current subject matter provides a better understanding of the preclinical and clinical response to these engineered cytokines. The QSP model described herein captures the PK, dynamic target mediated-drug disposition, cellular expansion, and target receptor expression across multiple physiologically relevant tissues in response to engineered IL-15 cytokine therapies in cynos and patients. The predictive capability of the QSP model has been calibrated and tested against multiple preclinical datasets from three different engineered IL-15 cytokine molecules, and can be extended to other cytokine molecules, such as those that drive lymphocyte proliferation. The QSP model described herein may additionally and/or alternatively be used to compare performance between each of the cytokine molecules. In particular, a greater drug exposure has been predicted in the terminal phase of the PK profile following treatment with Xmab24306 compared to PD1/IL-15 TaCk. Additionally, the experimental data disclosed herein suggest that while the fold-change of cells in the blood is reflective of dynamics in the tissue, Xmab24306 and PD1/IL-15 TaCk bind to differing cell populations in the blood but similar cell populations in the tissue. Finally, the QSP model described herein integrates preclinical PD1/IL-15 TaCk datasets as well as a clinical Xmab24306 dataset to make predictions about FIH dosing of PD1/IL-15 TaCk in a virtual cohort.

After making predictions about cell expansion and drug exposure within a virtual patient cohort, said cohort was used to explore variability in parameter space and find that both drug exposure and cellular expansion during PD1/IL-15 TaCk (e.g., cytokine molecule) treatment depends on the target receptor level in patients. In particular, based on the QSP model described herein, it has been predicted that CD8+ T cell (among other lymphocyte populations) fold changes within the tumor is intrinsically linked to the receptor expression at the surface of those cells: specifically, simulations with greater IL-15R and PD1 receptor expression on CD8+ cells that were PD1^{hi} were associated with >5 fold change in CD8+ T cell counts in the tumor.

This prediction has important consequences within the context of combination therapies with PD1/IL-15 TaCk (or other PD1-targeting molecules), as recent studies have shown, anti-PD1 therapies can lead to greater PD1 receptor expression on tumor-infiltrating cytotoxic T cells. Pretreatment with these therapies may be a technique to yield greater PD1 expression prior to PD1/IL-15 TaCk treatment, a combination approach that could lead to a greater fold change of these crucial cell types in the tumor.

There are a number of important observations. Most notably, the present disclosure highlights the sensitivity of the model to both IL-15 and PD1 receptor expression. However, the exact number of receptors at the cell surface needs to be quantified, as it can change across time as well as from subject to subject. Consistent with embodiments of the current subject matter, a combination of internal experimental data, literature reports, and model calibration has bee used to estimate the count of receptors per cell, but these estimates are perhaps best viewed as relative expression among the modeled cell-types rather than absolute expression levels. As is often the case with QSP and biological systems models, the QSP model disclosed herein is relatively large in scope and underspecified due to the mechanistic complexity relative to available data. As such, the use of this model focused on identifying a range of behaviors, comparing treatments with known parametric differences (e.g., molecular weight), and employing virtual cohort methodologies for the investigation of uncertainty around key parameters within the model, techniques which have previously proven successful in generating accurate predictions with underspecified models. Accordingly, the QSP model described herein can be used to make accurate predictions of distributions of lymphocytes in various tissue compartments of patients and cynos.

The model disclosed herein can have considerable applications beyond those presented herein. Currently, the QSP model serves as a formal integration of preclinical and clinical data from three separate engineered cytokines into a unified, quantitative explanation of the biology of lymphocyte expansion following IL-15 receptor agonism. In this instance, the QSP model described herein captures drug: receptor binding and subsequent target cell activation, proliferation, margination/trafficking, and depletion to describe the response of cynos and humans to cytokine molecules, such as PD1/IL-15 Tack, RSV IL-15 TaCk, and Xmab24306. The QSP model and its ability to recapitulate experimental data increase the confidence in the understanding of the mechanisms underlying lymphocyte expansion following treatment with engineered IL-15 cytokines. However, the QSP model could be extended to integrate data and make predictions about any other molecules that activate the immune system and display a dynamic PK PD relationship.

Overall, the QSP model described herein offers a novel approach in integrating preclinical and clinical datasets to aid the translation and dosing decisions of novel molecules. Further, this work highlights the power of systems biology approaches and the QSP model described herein: pairing experimental and QSP modalities is a critical approach to inform translational efforts and uncover the dynamic relationship between PK and PD when dosing with an agent that activates and stimulates the immune system. Based on the dynamic relationship between PK and PD provided by the QSP model described herein, the behavior of the lymphocytes over time in various compartments of a patient may be predicted, and treatment plans may be determined based on such predicted behaviors.

As described in greater detail herein, some embodiments of this disclosure relate to a novel QSP model that captures PK, dynamic target-mediated drug disposition (TMDD), cellular expansion, and target receptor expression in multiple tissues within a minPBPK modeling framework. This model is a fully coupled PKPD model in which the pool of targets (e.g., PD1 and IL-15R) is significantly expanded by drug exposure via cell (e.g., lymphocyte) proliferation. Subsequently, cell proliferation also affects PK through (TMDD).

The modeling framework described herein provides a novel approach to evaluate the competition among cells to bind to drug molecules (e.g., cytokine molecules) and how that subsequently translates to signal induction and cell proliferation. Furthermore, the modeling framework described herein provides a novel approach to evaluate lymphocyte expansion in different tissues versus tumors based on the target expression.

A non-limiting exemplary QSP model of the disclosure is described in Example 1, where PKPD data has been captured across multiple molecules (with different molecular properties), across multiple species (cynomolgus monkey and human) to develop a QSP model that can be useful in supporting preclinical, translational and early clinical development of therapeutic molecules targeting a cell signaling pathway of interest, e.g., IL-15-mediated pathway. For example, the QSP model described herein can be used to design other engineered IL-15 molecules with different targeting arms and different affinities. Additionally, this approach can be generalized to support other targeted engineered cytokines that act to activate the immune system, e.g., immunostimulatory cytokines.

In the following detailed description, reference is made to the accompanying drawings, which form a part hereof. In the drawings, similar symbols generally identify similar components, unless context dictates otherwise. The illustrative alternatives described in the detailed description, drawings, and claims are not meant to be limiting. Other alternatives may be used and other changes may be made without departing from the spirit or scope of the subject matter presented here. It will be readily understood that the aspects, as generally described herein, and illustrated in the Figures, can be arranged, substituted, combined, and designed in a wide variety of different configurations, all of which are explicitly contemplated and make part of this application.

### Definitions

Unless otherwise defined, all terms of art, notations, and other scientific terms or terminology used herein are intended to have the meanings commonly understood by those of skill in the art to which this application pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

The singular form “a”, “an”, and “the” include plural references unless the context clearly dictates otherwise. For example, the term “a cell” includes one or more cells, comprising mixtures thereof. “A and/or B” is used herein to include all of the following alternatives: “A”, “B”, “A or B”, and “A and B”.

The term “operably linked”,” as used herein, denotes a physical or functional linkage between two or more elements, e.g., polypeptide sequences or polynucleotide sequences, which permits them to operate in their intended fashion. For example, an operable linkage between a polynucleotide of interest and a regulatory sequence (for example, a promoter) is a functional link that allows for expression of the polynucleotide of interest. In this sense, the term “operably linked” refers to the positioning of a regulatory region and a coding sequence to be transcribed so that the regulatory region is effective for regulating transcription or translation of the coding sequence of interest. Thus, a promoter is in operable linkage with a nucleic acid sequence if it can mediate transcription of the nucleic acid sequence. It should be understood that operably linked elements may be contiguous or non-contiguous. In the context of a polypeptide, “operably linked” refers to a physical linkage (e.g., directly or indirectly linked) between amino acid sequences (e.g., different segments, modules, or domains) to provide for a described activity of the polypeptide. In the present disclosure, various segments, regions, or domains of the recombinant polypeptides (e.g., multivalent polypeptides and multivalent antibodies) of the disclosure may be operably linked to retain proper folding, processing, targeting, expression, binding, and other functional properties of the recombinant polypeptides in the cell. Unless stated otherwise, various modules, domains, and segments of the recombinant polypeptides of the disclosure are operably linked to each other. Operably linked modules, domains, and segments of the multivalent polypeptides or multivalent antibodies of the disclosure may be contiguous or non-contiguous (e.g., linked to one another through a linker).

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number. If the degree of approximation is not otherwise clear from the context, “about” means either within plus or minus 10% of the provided value, or rounded to the nearest significant figure, in all cases inclusive of the provided value. In some embodiments, the term “about” indicates the designated value±up to 10%, up to ±5%, or up to ±1%.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

### Cytokines

Cytokines are polypeptides or glycoproteins with a molecular weight usually below 30 kDa (e.g., ˜5-20 kDa) that provide growth, differentiation and inflammatory or anti-inflammatory signals to different cell types, and therefore are important in cell signaling. Cytokines are most often released during a defined period in response to a stimulus, and the extent of their action is short-lived due to their limited half-life in the circulation. As a result, cytokines normally exert an autocrine or paracrine effect. As an exception to the general rule, cytokines such as interleukin (IL)-7 or haematopoietic growth factors are produced homeostatically in a continuous fashion. Cytokine target cells express high-affinity receptors on their cellular membrane. Following cytokine binding, the receptors trigger intracellular signaling which leads to modifications in gene transcription. Cytokines thereby modify proliferation and differentiation and induce or modify particular cell functions. Target cells expressing the corresponding sets of receptors integrate the information derived from the concentration and timing of exposure to different cytokines. Thus, synergy or antagonism among different cytokines is a common characteristic, with high degrees of complexity.

Cytokines have been shown to be involved in autocrine, paracrine and endocrine signaling as immunomodulating agents. Cytokines mediate cellular activities in a number of ways. Cytokines support the proliferation, growth, and differentiation of pluripotential hematopoietic stem cells into vast numbers of progenitors comprising diverse cellular lineages making up a complex immune system. Proper and balanced interactions between the cellular components are necessary for a healthy immune response. The different cellular lineages often respond in a different manner when cytokines are administered in conjunction with other agents.

Cytokines include chemokines, interferons, interleukins, lymphokines, and tumour necrosis factors, but generally not hormones or growth factors. Cytokines are produced by a broad range of cells, including immune cells like macrophages, B lymphocytes, T lymphocytes and mast cells, as well as endothelial cells, fibroblasts, and various stromal cells; a given cytokine may be produced by more than one type of cell. They act through cell surface receptors and are especially important in the immune system; cytokines modulate the balance between humoral and cell-based immune responses, and they regulate the maturation, growth, and responsiveness of particular cell populations. Some cytokines enhance or inhibit the action of other cytokines in complex ways. They are different from hormones, which are also important cell signaling molecules. Hormones circulate in higher concentrations, and tend to be made by specific kinds of cells. Cytokines are important in health and disease, specifically in host immune responses to infection, inflammation, trauma, sepsis, cancer, and reproduction.

Cytokines are generally classified into four types: (1) the IL-2 subfamily which is the largest family. It contains several non-immunological cytokines including erythropoietin (EPO) and thrombopoietin (TPO). They can be grouped into long-chain and short-chain cytokines by topology. Some members share the common gamma chain as part of their receptor. (2) The interferon (IFN) subfamily. (3) The IL-10 subfamily; and (4) the IL-1 family, which primarily includes IL-1 and IL-18.

It is also reported that the cysteine knot cytokines (IPR029034) include members of the transforming growth factor beta superfamily, including TGF-β1, TGF-β2 and TGF-β3. In addition, the IL-17 family includes member cytokines that have a specific effect in promoting proliferation of T-cells that cause cytotoxic effects.

According to the Kyoto Encyclopedia of Genes and Genomes, cytokines can also be cataloged into 9 main groups: (1) chemokines, small cytokines with chemotactic activities that can further be subdivided into “C,” “CC,” “CXC” and “CX3C” chemokines, depending on the number and arrangement of conserved cysteine residues; (2) hematopoietic growth factors (or hematopoietins), i.e., cytokines with a prominent role in hematopoiesis, which can be further grouped-based on their respective receptors—into “gp130 (IL6ST) shared,” “IL13RA1 shared,” “IL12RB1 shared,” “IL3RB (CSF2RB) shared,” “ILRG shared” and “others”; (3) interleukin-1 family members; (4) interleukin-10 family members; (5) interleukin-17 family members; (6) interferons (IFNs); (7) platelet-derived growth factor (PDGF) family members; (8) transforming growth factor β (TGFβ) family members; and (9) tumor necrosis factors.

More information regarding the potential of cytokines in immunotherapy and particularly in clinical cancer immunotherapy can be found in, for example, Chulpanova D. S. et al., Front. Cell Dev. Biol., 3 Jun. 2020, and Berraondo P. et al., British Journal of Cancer volume 120, 6-15 (2019), both of which are incorporated by reference.

The molecular aspects of a number of exemplary cytokine-based cancer immunotherapies are described in more detail below.

### IL-2

IL-2 is a member of a cytokine family, each member of which has a four alpha helix bundle; the family also includes IL-4, IL-7, IL-9, IL-15 and IL-21. IL-2 signals through the IL-2 receptor, a complex consisting of three chains, termed alpha (CD25), beta (CD122) and gamma (CD132). The gamma chain is shared by all family members. The IL-2 receptor (IL-2R) α subunit binds IL-2 with low affinity (Kd˜10-8 M). Interaction of IL-2 and CD25 alone does not lead to signal transduction due to its short intracellular chain but has the ability (when bound to the β and γ subunit) to increase the IL-2R affinity 100-fold. Heterodimerization of the β and γ subunits of IL-2R is essential for signaling in T cells. IL-2 can signalize either via intermediate-affinity dimeric CD122/CD132 IL-2R (Kd˜10-9 M) or high-affinity trimeric CD25/CD122/CD132 IL-2R (Kd˜10-11 M). Dimeric IL-2R is expressed by memory CD8+ T cells and NK cells, whereas regulatory T cells and activated T cells express high levels of trimeric IL-2R.

Interleukin 2 (IL-2) is predominantly produced by antigen-stimulated CD4⁺ T-cells, as well as NKT-cells, CD8⁺ T-cells, mast cells and DCs. IL-2 can stimulate the proliferation of antigen-activated CD8⁺ T-cells, treatment with endogenous IL-2 leads to an increase in the expression of CD25, IL-2 receptor, which in turn stimulates the proliferation of CD8⁺ T-cells. IL-2 increases the expression of LAMP-1 on the surface of CD8⁺ T-cells, decreases the expression of PD-1, an immunosuppressive receptor, thereby mediating the cytotoxic activity of CD8⁺ T-cells. IL-2 can stimulate the expansion and activation of NK cells with CD56^{high} CD16⁻ receptor, with the cytotoxic function of this population increasing after activation. This cytokine also enhances the cytotoxic effect of γδ T-cells, increasing CD69 and degranulation marker CD107a expression and IFN-γ secretion. Furthermore, IL-2 can also promote the expansion of NKT-cells in cancer patients. IL-2-based therapy has several significant drawbacks, particularly the stimulation of Tregs, which are associated with the suppression of the antitumor immune response. Stimulation with IL-2 leads to increased expression of CD25, CTLA-4, and HLA-DR on the surface of CD3⁺ T-cells. IL-2 treatment leads to an increase in the expression of FOXP3 in CD3⁺CD25⁺ T-cells and the formation of the Tregs phenotype, including during low-dose therapy. However, recent discoveries in understanding the functioning of Tregs have opened up new possibilities for the use of IL-2 in combination with Treg inhibitors, such as anti-CTLA-4 and anti-PD-1. The combination of IL-12 and IL-21 has also been shown to block IL-2-induced Treg activation.

Currently, IL-2 has found its place in cancer immunotherapy for the expansion of immune cells such as NK cells, T-cells, NKT-cells, cytokine-induced killer (CIK) cells. IL-2 is also used as an adjuvant in the treatment of patients with melanoma, advanced colorectal cancer or ovarian cancer with autologous dendritic cells stimulated by autologous tumor lysate, as well as in the treatment with viral vaccines. The addition of IL-2 to the treatment regimen can increase the effectiveness of therapy due to the induction of expansion of tumor antigen presented T-cell.

IL-2 is also effective to treat patients with melanoma when combined with dacarbazine monotherapy or anti-VEGF monoclonal antibody (mAb) monotherapy, but do not increase the effectiveness of neuroblastoma therapy in combination with dinutuximab (anti-GD2 mAb). Several clinical trials have also evaluated the effectiveness of the combination of radiotherapy and IL-2.

### IL-12

Interleukin 12 is another cytokine with well-studied antitumor activity, which is mainly mediated by stimulation of IFN-γ production in cytotoxic cells (CD8 T-cells and NK cells) and Th1 cells. IL-12 is naturally produced by dendritic cells, macrophages, neutrophils, and human B-lymphoblastoid cells (NC-37) in response to antigenic stimulation. IL-12 belongs to the family of interleukin-12. IL-12 family is unique in comprising the only heterodimeric cytokines, which includes IL-12, IL-23, IL-27 and IL-35.

IL-12 binds to the IL-12 receptor, which is a heterodimeric receptor formed by IL-12Rβ1 and IL-12Rβ2. IL-12Rβ2 is considered to play a key role in IL-12 function, since it is found on activated T cells and is stimulated by cytokines that promote Th1 cells development and inhibited by those that promote Th2 cells development. Upon binding, IL-12R-β2 becomes tyrosine phosphorylated and provides binding sites for kinases, Tyk2 and Jak2. These are important in activating critical transcription factor proteins such as STAT4 that are implicated in IL-12 signaling in T cells and NK cells. This pathway is known as the JAK-STAT pathway.

Treatment with IL-12 leads with varying degrees of success to an increase in the number of CD56⁺ NK cells, as well as CD2 and LFA-1 expression, which ultimately results in the increased cytotoxicity of IL-12-treated NK cells. IL-12 also stimulates the production of IFN-γ in cytotoxic CD8⁺ T-cells, enhances their proliferative activity and cytotoxicity, probably due to increased expression of GrzmB. In addition, IL-12-stimulated CD8 T-cells are able to reduce the number of Tregs in TME by Fas-mediated apoptosis. IL-12 is also involved in the differentiation of naïve Th cells in Th1 cells. Promising results in cell and animal models have prompted clinical studies of IL-12. However, the future of IL-12 in the treatment of cancer was overshadowed by the significant side effects that were observed in the first clinical trials. The established protocol in the Phase I study schedule of IL-12 administration was slightly amended that led to the development of severe IFN-γ-mediated toxicity, resulting in 12 patients being hospitalized and two patients dying. Clinical trials of IL-12 have continued, but with more caution, however, ultimately IL-12 has not been shown particular efficacy, either alone or in combination with various therapeutic agents, with the exception of patients with cutaneous T-cell lymphoma (CTCL), with non-Hodgkin's B-cell lymphoma, and with acquired immune deficiency syndrome (AIDS)-associated Kaposi sarcoma.

### IL-15

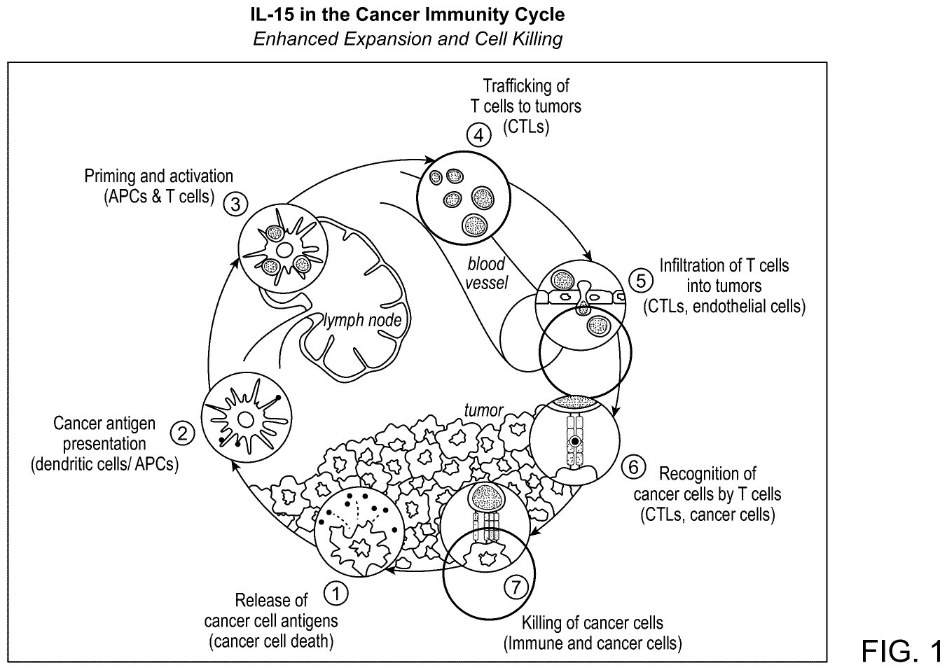

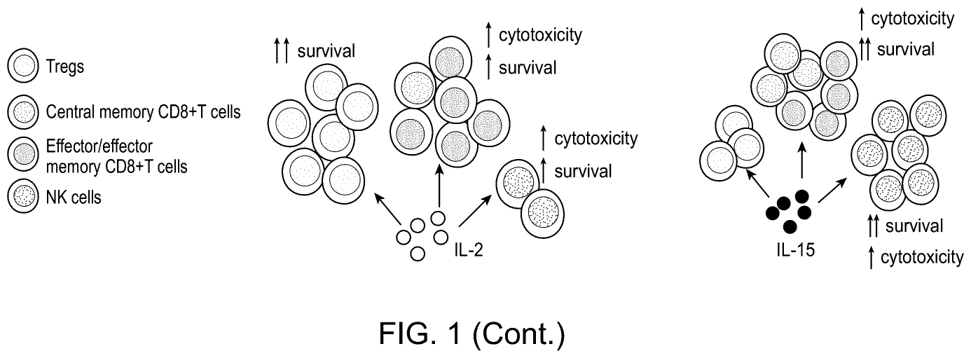

Interleukin-15 (IL-15) is another cytokine that belongs to the same family as IL-2 and has many overlapping functions. IL-15 is involved in the stimulation of cytolytic activity, cytokine secretion, proliferation and survival of NK cells, CD8+ memory T-cells and naïve CD8+ cells (see, e.g., FIG. 1). As a pleiotropic cytokine, it plays important roles in innate and adaptive immunity. IL-15 is constitutively expressed by a large number of cell types, including macrophages, monocytes, dendritic cells and fibroblasts. Expression of IL-15 can be stimulated by, for example, cytokines (e.g., GM-CSF), double-stranded mRNA, unmethylated CpG oligonucleotides, lipopolysaccharide through Toll-like receptors, and interferons (e.g., IFN-γ), or after infection of, for example, monocytes with herpes virus, Mycobacterium tuberculosis and Candida albicans.

IL-15 binds to a specific receptor complex on T-cells and NK cells. IL-15 and IL-15Rα are co-expressed on activated dendritic cells and on monocytes, and IL-15 functions in a complex with IL-15Rα. IL-15/IL-15α bind as a heterodimer to two chains on T-cells and NK cells-IL-2Rβ (also referred to as IL-15Rβ or CD122) and γc (also referred to as IL-2RG; CD132; γ-c; or common γ-chain) molecules. The β and γc chains are shared between IL-2 and IL-15 and are essential for the signaling of these cytokines.

Consistent with the sharing of the IL-2/IL-15βγc receptor complex, IL-15 has been shown to mediate many functions similar to those of IL-2 in vitro. They share many biological activities and exhibit similar contributions to the survival of T lymphocytes. It is believed that the biological differences between IL-2 and IL-15 are likely due to, for example, their different production sites, their strength of association with membrane receptor proteins, termed IL-2α and IL-15Rα, respectively, and the regulation of these extra receptor molecules. IL-2 and IL-15 play a role in regulating the number of CD8+ memory cells.

### IL-21

Interleukin 21, another member of the IL-2 family, has been investigated for the clinical use in cancer treatment. IL-21 has potent regulatory effects on cells of the immune system, including natural killer (NK) cells and cytotoxic T cells that can destroy virally infected or cancerous cells. This cytokine induces cell division/proliferation in its target cells.

The IL-21 receptor (IL-21R) is expressed on the surface of T, B and NK cells. IL-21R is similar in structure to the receptors for other type I cytokines like IL-2R or IL-15 and requires dimerization with the common gamma chain (γc) in order to bind IL-21. When bound to IL-21, the IL-21 receptor acts through the Jak/STAT pathway, utilizing Jak1 and Jak3 and a STAT3 homodimer to activate its target genes.

One of the most important functions of IL-21 is to stimulate the proliferation of germinal center (GC) B cells, as well as to induce the differentiation CD40L-stimulated B cells in plasma cells. However, IL-21 treatment also results in an increase in the number of B10 cells as well as the levels of IL10 that they produce. The increase in IL10 secretion after IL-21 stimulation is also observed in CD4⁺, CD8⁺ T-cells. IL-21 is also able to activate NK cells by stimulating the expression of CD69 and the natural cytotoxicity receptor NKp46 and increasing cytotoxic activity. IL-21 stimulates differentiation of naïve CD4⁺ T-cells in Th17 cells, inducing expression of IL17, retinoic-acid-receptor-related orphan nuclear receptor gamma (RORγt) transcription factor and IL-23R. Also, this cytokine plays an important role in the autocrine stimulation of proliferation and differentiation (expression of inducible T-cell costimulator (ICOS) is increased) of Tfh. It is reported that IL-21 negatively regulates homeostasis of CD4⁺ CD25⁺ FOXP3⁺ Tregs. However, in clinical trials the therapeutic effect of IL-21 is evaluated by analyzing the secretion of soluble CD25, the level of which increases during the activation of T-cells and NK cells.

Clinical trials have shown that rIL-21 is able to increase the number of CD3⁺ CD56 NKT-like cells in patients with stage IV malignant melanoma, and also activate T and NK cells in patients with stage IV colorectal cancer. The combination of IL-21 with various mAbs showed that the combination of IL-21 with rituximab (anti-CD20 mAb) or sorafenib (anti-VEGF mAb) was well tolerated and had antitumor activity. However, the contribution of IL-21 into the shown antitumor activity has not been fully determined.

Interleukin 21, like other members of its family, is used to activate and expand T-cell and NK cells. There are several completed and active clinical trials where IL-21 is combined with checkpoint inhibitors or with anti-CD19 CAR T-cells.

### Interferons

Another group of cytokines, interferons, has also been shown to be effective in cancer immunotherapy. IFNs are divided into three types depending on the function and the target receptor: type I (α, β, ε, κ, and ω), type II (γ), and type III (λ). IFN-α2a was the first approved cytokine for the treatment of chronic myeloid leukemia, since type I IFNs have broad immunomodulatory activity. IFN-α can stimulate differentiation of CD14⁺ monocytes in DCs jointly with GM-CSF. IFN-α/GM-CSF stimulation leads to the increased expression of HLA-DR, CD11c, CD83, B7 costimulatory molecules CD80 and CD86, including on DCs of cancer patients, and such DCs are able to efficiently present an antigen to CD4⁺ and CD8⁺ T-cells. However, IFN-β reduces the ability of mature DCs to stimulate T-cell proliferation and differentiate into IFN-γ-producing Th1 cells. In addition to its apparent effect on T-cells via DCs, IFN-α is also able to stimulate the effector functions of pre-activated CD8⁺ T-cell (which have already interacted with antigen and costimulatory molecules), leading to increased number of activation markers CD38 and CD25 and raised expression of GrzmB, TRAIL, FasL, and IFN-γ. Type I IFNs have different effects on the differentiation of CD4⁺ T-cells, supporting the polarization into Th1 cells and inhibiting the formation of Th2 and Th17 T-cell phenotypes. Type I IFNs also play an important role in the regulation of NK cell cytotoxicity, however, low cytotoxicity in the absence of type I IFN stimulation can be overcomed by stimulation with IL-2.

IFN-γ is a type II IFN cytokine which can both regulate the antitumor immune response and directly induce apoptosis of tumor cells. In regards to the regulation of the immune system cells, it was shown that IFN-γ therapy leads to a significant increase in the number of CD14^{high} CD16⁺ monocytes and a rise in MHCII expression on all monocytes in patients with various types of tumors. The number of activated NK cells also increases (expression of NK receptor activation marker NKp30 was increased on both CD56^{high} and CD56^{low} NK cells). In addition, a number of investigations in mouse models has been shown that IFN-γ is able to modulate polarization toward CD86⁺ iNOS⁺ MI macrophages, which can inhibit tumor cell growth by releasing NO. Nevertheless, IFN-γ stimulates differentiation of CD4⁺CD25⁻ T-cells in CD4⁺ Tregs in a mouse model of experimental autoimmune encephalomyelitis as well inhibiting the proliferation of Th2 cells. However, the effect of IFN-γ on various T-cell populations in cancer patients requires more detailed investigation. In addition, there is evidence of the pro-tumor effect of IFN-γ which also requires the attention of researchers.

Type I interferons are actively combined with various therapeutic agents in clinical trials (NCT03112590). In the number of clinical trials, IFN-α or IFN-β were used to stimulate the immune response in patients who received therapy with DC vaccinesor tumor-specific antigens. Vaccination itself did not always lead to the increase in OS or showed some encouraging results. However, IFN-α administration was sometimes able to significantly increase OS compared to a single vaccine.

Interferons have also been combined with chemotherapy and mAbs for the treatment of various types of tumors. The combination of IFN-β with temozolomide did not show any promising results in patients with glioblastoma. Also, the combination of IFNs with bevacizumab (anti-VEGF mAb) did not show much benefit compared to the combination of bevacizumab+everolimus, which is usually used to treat metastatic renal cell carcinoma.

In general, there are not many clinical trials that are ongoing to evaluate the efficacy of combination of IFNs with immune checkpoint inhibitors, most often IFN-α monotherapy is used as a reference to evaluate the effectiveness of the inhibitors for the treatment of advanced melanoma. However, the clinical trials of IFN-α and pembrolizumab have shown the safety but had limited antitumor activity of the IFN-α+pembrolizumab combination. Several clinical trials of IFN-γ with PD-1 inhibitors are ongoing.

### Quantitative System Pharmacology

As described above, significant scientific advances in pharmacometrics suggest that pharmacometric analyses improve the efficiency of the drug development process by simulating complex and diverse data drug information for populations or clinical scenarios that are difficult to test. The QSP model described herein may be used to: (1) aid in the design of clinical trials, (2) identify clinically relevant covariates, (3) characterize pharmacokinetic and exposure-response profiles, (4) design molecules, 5) translate preclinical to clinical data, and (6) extrapolate efficacy to special populations, for example, pediatrics or geriatrics.

Using pharmacometrics can provide efficient and cost-effective approaches to support and optimize drug safety and effectiveness. For example, quantitative system pharmacology (QSP) models, such as the QSP model described herein, which link PK with mechanistic models of biological pathway modulation, can be very useful. The QSP model described herein enables the separation of biological and drug specific parameters, and thus has an enhanced interspecies translational ability. This property is ideally suited for translational modeling of immuno-oncology agents since the QSP model can be re-parametrized using human-specific biological parameters while keeping the drug-specific parameters the same between mice and humans since these are properties of the drug.

QSP combines computational models of systems biology and systems pharmacology. With the development of high-throughput techniques (genomics, transcriptomics, proteomics, and metabolomics) as well as computer and bioinformatics methods, systems biology and systems pharmacology modeling has been increasingly used to comprehend human biology and disease progression, predict the effectiveness and safety of drug candidates. Examples of QSP applications include uses of mathematical computer models to characterize biological systems, disease processes and drug pharmacology. QSP can be viewed as a sub-discipline of pharmacometrics that focuses on modeling the mechanisms of drug pharmacokinetics (PK), pharmacodynamics (PD), and disease processes using a systems pharmacology point of view. QSP models are typically defined by systems of ordinary differential equations (ODE) that depict the dynamical properties of the interaction between the drug and the biological system.

The early intervention and full participation of QSP in the development of new drugs discovery can form a model-led drug development model to improve the efficiency of drug discovery and scientific approval, reduce the cost of research and development, and shorten the time to market for new drugs.

QSP can be used to generate biological/pharmacological hypotheses in silico to aid in the design of in vitro or in vivo non-clinical and clinical experiments. This can help to guide biomedical experiments so that they yield more meaningful data. QSP is increasingly being used for this purpose in pharmaceutical research and development to help guide the discovery and development of new therapies QSP has been used by the FDA in a clinical pharmacology review.

The QSP model described herein can be used to evaluate molecules of interest, such as cytokines (e.g., immunostimulatory cytokines), for its ability to expand certain types of immune cells in multiple compartments. The QSP model described herein may additionally and/or alternatively be employed to determine effective treatment plans including effective doses for the cytokine molecules, predict distributions of lymphocytes in various physiological compartments of a patient over time, and/or the like.

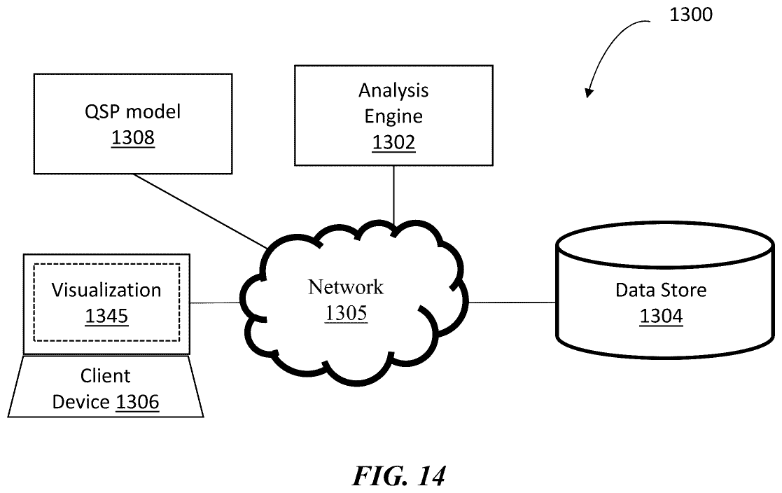

FIG. 14 depicts a system diagram illustrating an example of an analysis system 1400, in accordance with some example embodiments. Referring to FIG. 14, the analysis system 1400 may include an analysis engine 1402 (e.g., a modeling engine), a data store 1404, a client device 1406, and a QSP model 1408. As shown in FIG. 14, the analysis engine 1402, the data store 1404, the client device 1406, and the QSP model 1408 may be communicatively coupled via a network 1405. The network 1405 may be a wired network and/or a wireless network including, for example, a local area network (LAN), a virtual local area network (VLAN), a wide area network (WAN), a public land mobile network (PLMN), the Internet, and/or the like. In some implementations, the analysis engine 1402, the QSP model 1408, the data store 1404, and/or the client device 1406 may be contained within and/or operate on a same device. It should be appreciated that the client device 1406 may be a processor-based device including, for example, a smartphone, a tablet computer, a wearable apparatus, a virtual assistant, an Internet-of-Things (IoT) appliance, and/or the like. The analysis engine 1402 includes at least one data processor and at least one memory storing instructions, which when executed by the at least one data processor, perform one or more operations as described herein. The client device 1406 may display an output of the QSP model 1408 via the visualization 1445. The data store 1404 may store input clinical data and/or one or more determinations made by the analysis engine 1402.

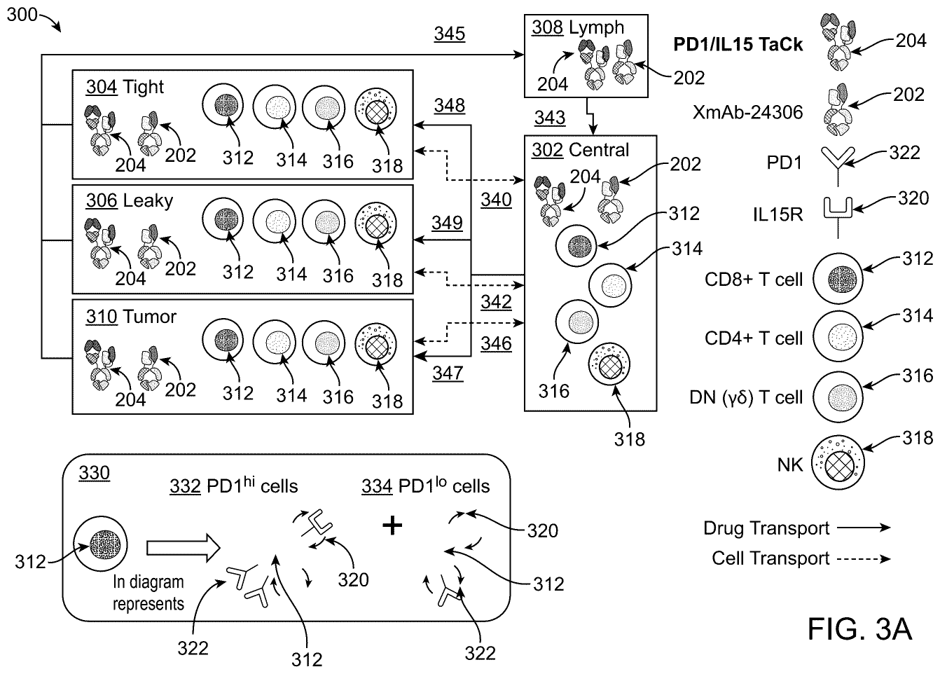

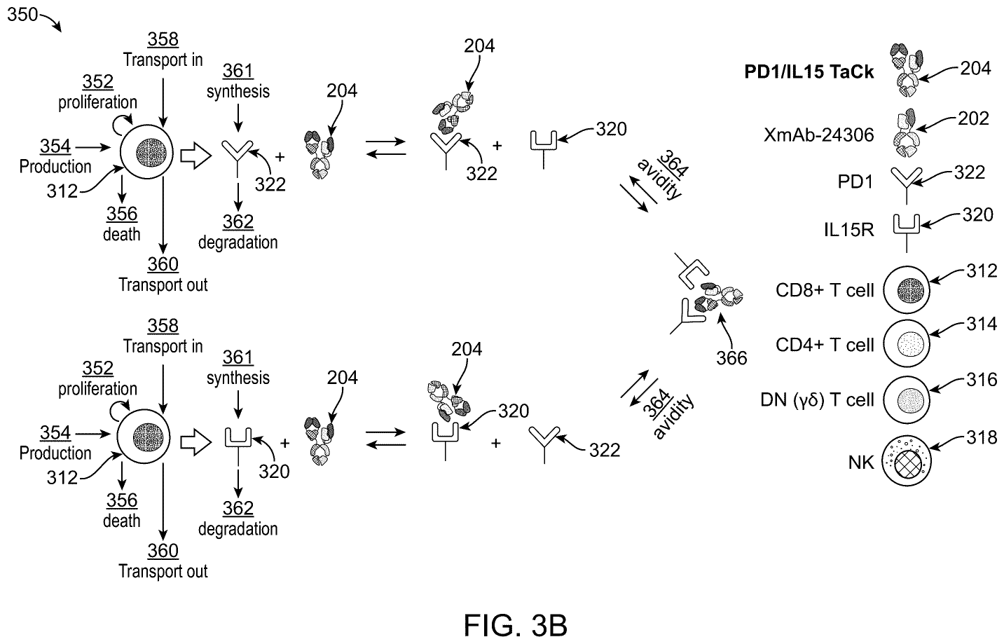

FIG. 3A schematically depicts an example architecture 300 of the QSP model 1408, consistent with embodiments of the current subject matter. As noted, the QSP model 1408 describes in vivo dynamics of drug: receptor binding and lymphocyte expansion following treatment with a cytokine molecule consistent with embodiments of the current subject matter.

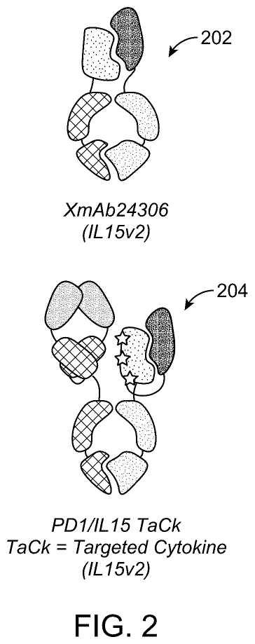

The cytokine molecule may be any cytokine that stimulates an immune system of the patient and/or proliferates expansion of the lymphocytes described herein. For example, the cytokine molecule may include interleukin 2 (IL-2), interleukin 7 (IL-7), interleukin 12 (IL-12), interleukin 15 (IL-15), interleukin 21 (IL-21), or interferon, as described herein. Additionally and/or alternatively, the cytokine molecule may include Xmab24306, PD1/IL15 TaCk, or RSV IL-15 TaCk. The Xmab24306, the PD1/IL15 TaCk, and/or the RSV IL-15 TaCk may each include at least one of the IL-2, IL-7, IL-12, IL-15, IL-21, and interferon. An example of the Xmab24306 (represented as 202) and the PD1/IL15 TaCk (represented as 204) is shown in FIG. 2. Additionally and/or alternatively, the cytokine molecule may include an engineered cytokine that agonizes (e.g., targets) its cognate receptor, such as a corresponding interleukin receptor (e.g., IL-15 receptor, and/or the like).

In an example, the cytokine molecule is a bispecific molecule including a first arm and a second arm. Interleukin 15 receptors (IL-15R) of each of the target lymphocytes bind to the first arm of the bispecific molecule, and PD1 receptors of each of the target lymphocytes bind to the second arm of the bispecific molecule. As another example, the cytokine molecule may be a multivalent polypeptide including a first polypeptide region capable of binding to a first target, and a second polypeptide region capable of binding to a second target. In this example, the first polypeptide region is operably linked to the second polypeptide region. Here, the first target may be a first receptor (e.g., IL-15R) of the cytokine molecule, and the second target may be a second receptor (e.g., PD1) of the cytokine molecule. A binding affinity of the second polypeptide region to the second target may be increased via avidity when the first polypeptide region binds to the first target. Further, a binding of the second polypeptide region to the second target has K_{d} of second interaction that is higher when the first polypeptide region binds to the first target. In some embodiments, the first target is a cytokine or a cytokine receptor, and the second target includes a natural ligand of the cytokine.

In some embodiments, at least one of the first and the second polypeptide regions includes a binding moiety that binds to the target molecule, e.g., a cytokine, a cytokine receptor, or a ligand of the cytokine. In some embodiments, the binding moiety includes one or more antigen-binding determinants of an antibody or a functional antigen-binding fragment thereof. Blocking antibodies and non-blocking antibodies are both suitable. A “blocking” antibody or an “antagonist” antibody refers to an antibody that prevents, inhibits, blocks, or reduces biological or functional activity of the antigen to which it binds. Blocking antibodies or antagonist antibodies can substantially or completely prevent, inhibit, block, or reduce the biological activity or function of the antigen.

An “antigen-binding fragment” generally refers to an antibody fragment such as, for example, a diabody, a Fab, a Fab′, a F(ab′)2, an Fv fragment, a disulfide stabilized Fv fragment (dsFv), a (dsFv) 2, a bispecific dsFv (dsFv-dsFv′), a disulfide stabilized diabody (ds diabody), a single-chain antibody molecule (scFv), an scFv dimer (e.g., bivalent diabody-bi-scFv or divalent diabody-di-scFv), or a multispecific antibody formed from a portion of an antibody including one or more complementarity-determining regions (CDRs) of the antibody. The antigen-binding moiety can include naturally-derived polypeptides, antibodies produced by immunization of a non-human animal, or antigen-binding moieties obtained from other sources (e.g., camelids.) The antigen-binding moiety can be engineered, synthesized, designed, humanized, or modified so as to provide desired and/or improved properties.

In some embodiments, the cytokine molecule is a polypeptide. In some embodiments, the cytokine molecule is a multivalent polypeptide. In some embodiments, the multivalent polypeptide of the disclosure is a multivalent antibody (e.g., bivalent antibody or trivalent antibody) including at least two binding moieties each possessing specific binding for a target protein. In some embodiments, the binding moieties possess specific binding for the same target protein. Such antibody is multivalent, monospecific antibody. In some embodiments, the binding moieties possessing specific binding for at least two different target proteins. Such antibody is multivalent, multispecific antibody (e.g., bispecific, trispecific, etc.) Accordingly, some embodiments disclosed herein relate to a multivalent antibody or functional fragment thereof, which includes (i) a first polypeptide region specific for cytokine or cytokine receptor, and (ii) a second polypeptide region specific for a ligand of the cytokine, wherein the first polypeptide region is operably linked to the second polypeptide region. Accordingly, in some embodiments, the disclosed multivalent antibody can be a bivalent, monospecific antibody. In some embodiments, the disclosed multivalent antibody can be a trivalent, monospecific antibody. In some embodiments, the disclosed multivalent antibody can be a bivalent, bispecific antibody.

The binding of a first polypeptide region and a second polypeptide region to their respective target can be either in a competitive or non-competitive fashion with a natural ligand of the target. Accordingly, in some embodiments of the disclosure, the binding of a first polypeptide region and/or second polypeptide region of the molecule of interest to their respective target can be ligand-blocking. In some other embodiments, the binding of a first polypeptide region and/or second polypeptide region to their respective target does not block binding of the target's natural ligand. As used herein, the term “multivalent polypeptide” as used herein refers to a polypeptide comprising two or more target-binding regions that are operably linked to each other. For example, a “bivalent” polypeptide of the disclosure comprises two target-binding regions, whereas a “trivalent” polypeptide of the disclosure comprises three target-binding regions. The amino acid sequences of the polypeptide regions may normally exist in separate proteins that are brought together in the multivalent polypeptide or they may normally exist in the same protein but are placed in a new arrangement in the multivalent polypeptide. A multivalent polypeptide may be created, for example, by chemical synthesis, or by creating and translating a polynucleotide in which the peptide regions are encoded in the desired relationship.

The binding activity of a molecule, e.g., multivalent polypeptide and multivalent antibody of the disclosure, to its target can be assayed by any suitable method known in the art. For example, the binding activity of the multivalent polypeptides and multivalent antibodies of the disclosure can be determined by, e.g., Scatchard analysis. Specific binding may be assessed using techniques known in the art including but not limited to competition ELISA, BIACORE® assays and/or KINEXA® assays. A variety of assay formats may be used to select an antibody or polypeptide that specifically binds a cytokine molecule. For example, solid-phase ELISA immunoassay, immunoprecipitation, Biacore™ (GE Healthcare, Piscataway, NJ), KinExA, fluorescence-activated cell sorting (FACS), Octet™ (ForteBio, Inc., Menlo Park, CA) and Western blot analysis are among many assays that may be used to identify a polypeptide of an antibody that specifically reacts with an antigen or a receptor, or ligand binding portion thereof, that specifically binds with a cognate ligand or binding partner.

Designation of the polypeptide region of the polypeptide of interest, e.g., multivalent polypeptide that is capable of binding to a first target, e.g., a cytokine or a cytokine receptor, as the “first” polypeptide region and the polypeptide region of the multivalent polypeptide capable of binding to second target, e.g., a natural ligand of the cytokine, as the “second” polypeptide region is not intended to imply any particular structural arrangement of the “first” and “second” polypeptide regions within the multivalent polypeptide. By way of non-limiting example, in some embodiments of the disclosure, the multivalent polypeptide or multivalent antibody may include (i) an N-terminal polypeptide region capable of binding to a cytokine or a cytokine receptor and (ii) a C-terminal polypeptide region including a polypeptide region capable of binding to a natural ligand of the cytokine. In other embodiments, the multivalent polypeptide or multivalent antibody may include an N-terminal polypeptide region capable of binding to a natural ligand of the cytokine and a C-terminal polypeptide region capable of binding to a cytokine or a cytokine receptor. In addition or alternatively, the multivalent polypeptide or multivalent antibody may include more than one polypeptide region capable of binding to a cytokine or a cytokine receptor, and/or more than one polypeptide region capable of binding to a natural ligand of the cytokine. Accordingly, in some embodiments, a first amino acid sequence of the multivalent polypeptide or multivalent antibody includes at least two, three, four, five, six, seven, eight, nine, or ten polypeptide regions each capable of binding to a cytokine or a cytokine receptor. In some embodiments, the at least two, three, four, five, six, seven, eight, nine, or ten polypeptide regions of a first amino acid sequence are each capable of binding to the same cytokine or a cytokine receptor. In some embodiments, the at least two, three, four, five, six, seven, eight, nine, or ten polypeptide regions of a first amino acid sequence are each capable of binding to different cytokines or a cytokine receptors. In some embodiments, a second amino acid sequence of the multivalent polypeptide or multivalent antibody includes at least two, three, four, five, six, seven, eight, nine, or ten polypeptide regions each capable of binding to a natural ligand of the receptor. In some embodiments, the at least two, three, four, five, six, seven, eight, nine, or ten polypeptide regions of a second amino acid sequence are each capable of binding to the same a natural ligand of the receptor. In some embodiments, the at least two, three, four, five, six, seven, eight, nine, or ten polypeptide regions of a first amino acid sequence are each capable of binding to different natural ligands of the receptor.

In some embodiments, a first polypeptide region of the multivalent polypeptide or multivalent antibody is directly linked to a second polypeptide region. In some embodiments, a first polypeptide region is directly linked to a second polypeptide region via at least one covalent bond. In some embodiments, a first polypeptide region is directly linked to a second polypeptide region via at least one peptide bond. In some embodiments, the C-terminal amino acid of a first polypeptide region can be operably linked to the N-terminal amino acid of a second polypeptide region. Alternatively, the N-terminal amino acid of a polypeptide region can be operably linked to the C-terminal amino acid of a second polypeptide region.

In some embodiments, a first polypeptide region of the multivalent polypeptide or multivalent antibody is operably linked to a second polypeptide region via a linker. There is no particular limitation on the linkers that can be used in the multivalent polypeptides described herein. In some embodiments, the linker is a synthetic compound linker such as, for example, a chemical cross-linking agent. Non-limiting examples of suitable cross-linking agents that are commercially available include N-hydroxysuccinimide (NHS), disuccinimidylsuberate (DSS), bis(sulfosuccinimidyl) suberate (BS3), dithiobis(succinimidylpropionate) (DSP), dithiobis(sulfosuccinimidylpropionate) (DTSSP), ethyleneglycol bis(succinimidylsuccinate) (EGS), ethyleneglycol bis(sulfosuccinimidylsuccinate) (sulfo-EGS), disuccinimidyl tartrate (DST), disulfosuccinimidyl tartrate (sulfo-DST), bis [2-(succinimidooxycarbonyloxy ethyl]sulfone (BSOCOES), and bis [2-(sulfosuccinimidooxycarbonyloxy)ethyl]sulfone (sulfo-BSOCOES).

In some embodiments, a first polypeptide region of a multivalent polypeptide or multivalent antibody disclosed herein is operably linked to a second polypeptide region via a polypeptide linker (peptidal linkage). In some embodiments, the polypeptide linker comprising a single-chain polypeptide sequence comprising about one to 100 amino acid residues (e.g., 2, 3, 4, 5, 6, 7, 8, 9, 10, 11, 12, 13, 14, 15, 16, 17, 18, 19, 20, etc. amino acid residues) can be used as a polypeptide linker. In some embodiments, the linker polypeptide sequence includes about 5 to 50, about 10 to 60, about 20 to 70, about 30 to 80, about 40 to 90, about 50 to 100, about 60 to 80, about 70 to 100, about 30 to 60, about 20 to 80, about 30 to 90 amino acid residues. In some embodiments, the linker polypeptide sequence includes about 1 to 10, about 5 to 15, about 10 to 20, about 15 to 25, about 20 to 40, about 30 to 50, about 40 to 60, about 50 to 70 amino acid residues. In some embodiments, the linker polypeptide sequence includes about 40 to 70, about 50 to 80, about 60 to 80, about 70 to 90, or about 80 to 100 amino acid residues. In some embodiments, the linker polypeptide sequence includes about 1 to 10, about 5 to 15, about 10 to 20, about 15 to 25 amino acid residues.

In some embodiments, the first polypeptide region is capable of binding to IL-15 receptor (CD122) or variant thereof. In some embodiments, the first target is IL-15R or a variant thereof. In some embodiments, the second polypeptide region is capable of binding to PD1 or a variant thereof. In some embodiments, the second target is PD1 or a variant thereof.

In some embodiments, the cytokine molecule is configured such that the binding avidity of the second region to the second target is increased when the first region binds to the first target. In some embodiments, the binding avidity of the second region to the second target is increased by at least 20%, e.g., at least 25%, at least 30%, at least 40%, at least 50%, at least 60%, at least 70%, at least 80%, at least 90%, or at least 95% when the first region binds to the first target. In some embodiments, the binding avidity of the second region to the second target is increased from 20% to 95%, e.g., from 20% to 50%, from 30% to 60%, from 40% to 80%, from 50% to 95%, from 25% to 70%, from 40% to 60%, from 30% to 80%, from 40% to 95%, or from 60% to 95%, when the first region binds to the first target. In some embodiments, the cytokine molecule is configured such that the binding of the second region to the second target has K_{d} of second interaction that is higher when the first region binds to the first target. In some embodiments, the cytokine molecule is configured such that the binding of the second region to the second target has K_{d} of second interaction that is at least 20%, e.g., at least 25%, at least 30%, at least 40%, at least 50%, at least 60%, at least 70%, at least 80%, at least 90%, or at least 95% higher when the first region binds to the first target. In some embodiments, the cytokine molecule is configured such that the binding of the second region to the second target has K_{d} of second interaction that is about 20% to 95%, e.g., 20% to 50%, 30% to 60%, 40% to 80%, 50% to 95%, 25% to 70%, 40% to 60%, 30% to 80%, 40% to 95%, or 60% to 95% higher when the first region binds to the first target.

Referring back to FIG. 3A, the architecture 300 of the QSP model 1408 includes a plurality of physiological compartments. For example, the architecture 300 includes a central compartment 302, a tight compartment 304, a leaky compartment 306, a lymph compartment 308, and a tumor compartment 308. The tight compartment 304 may include muscle, skin, adipose, and/or brain tissue of a subject (e.g., a patient, a cyno, etc.). The leaky compartment 306 may include a liver, a kidney, a heart, a lung, a spleen, a live nude, a bone, and/or an intestine of the subject. The tumor compartment 308 may be included for clinical simulations. The tumor compartment 308 may include a site of a tumor configured to be treated by the cytokine molecule. The particular physiological compartments included in the architecture 300 were selected to improve the accuracy of predictions generated based on the QSP model 1408, to more accurately track lymphocyte behavior within and between the plurality of physiological compartments, and to more accurately determine distributions of the lymphocytes. Thus, in some implementations, the plurality of physiological compartments included in the QSP model 1408 are only and/or all of the central compartment 302, the tight compartment 304, the leaky compartment 306, and the lymph compartment 308. In other implementations, the plurality of physiological compartments included in the QSP model 1408 are only the central compartment 302, the tight compartment 304, the leaky compartment 306, the lymph compartment 308, and the tumor compartment 308.

The QSP model 1408 may track at least one lymphocyte population corresponding to at least one target lymphocyte. The at least one target lymphocyte is targeted by the cytokine molecule. A behavior of the at least one target lymphocyte after delivery of the cytokine to the subject may be determined based on the QSP model 1408. The at least one target lymphocyte may include a plurality of target lymphocytes. The plurality of target lymphocytes includes a CD8+ T cell 312, a CD4+ T cell 314, a CD4−/CD8− (Double Negative) T cell (e.g., a gamma delta (gd) T cell) 316, and a natural killer (NK) cell 318. The particular target lymphocytes of the plurality of target lymphocytes included in the architecture 300 were selected to improve the accuracy of predictions generated based on the QSP model 1408, to more accurately track lymphocyte behavior within and between the plurality of physiological compartments, and to more accurately determine distributions of the lymphocytes. Thus, in some implementations, the plurality of target lymphocytes included in the QSP model 1408 are only and/or all of the CD8+ T cell 312, the CD4+ T cell 314, the CD4−/CD8−(Double Negative) T cell 316, and the natural killer (NK) cell 318.

The plurality of target lymphocytes (e.g., the CD8+ T cell 312, the CD4+ T cell 314, the CD4−/CD8−(Double Negative) T cell 316, and the natural killer (NK) cell 318) may each include a first subpopulation associated with high PD1 expression (PD1^{hi} cells) and a second subpopulation associated with low PD1 expression (PD1^{lo} cells). For example, as shown at 330 in FIG. 3A, the CD8+ T cell 312 includes a first subpopulation of PD1^{hi} cells 332 and a second subpopulation of PD1^{lo} cells 334. While only the CD8+ T cell 312 is shown in FIG. 3A as having the first subpopulation of PD1^{hi} cells 332 and the second subpopulation of PD1^{lo} cells 334, the CD4+ T cell 314, the CD4−/CD8− (Double Negative) T cell 316, and/or the natural killer (NK) cell 318 may include the first subpopulation of PD1^{hi} cells 332 and the second subpopulation of PD1^{lo} cells 334.

Additionally and/or alternatively, In some embodiments, the plurality of target lymphocytes includes B cells, monocytes, natural killer (NK) cells, natural killer T (NKT) cells, basophils, eosinophils, neutrophils, dendritic cells, macrophages, regulatory T cells, helper T cells (T_{H}), cytotoxic T cells (T_{CTL}), memory T cells, gamma delta (γδ) T cells (e.g., double negative T cell), hematopoietic stem cells, and/or hematopoietic stem cell progenitors.

In some embodiments, the plurality of target lymphocytes include a T lymphocyte or a T lymphocyte progenitor. In some embodiments, the T lymphocyte is a CD4+ T cell or a CD8+ T cell. In some embodiments, the T lymphocyte is a CD8+T cytotoxic lymphocyte cell selected from the group consisting of naïve CD8+ T cells, central memory CD8+ T cells, effector memory CD8+ T cells, effector CD8+ T cells, CD8+ stem memory T cells, and bulk CD8+ T cells. In some embodiments, the T lymphocyte is a CD4+T helper lymphocyte cell selected from the group consisting of naïve CD4+ T cells, central memory CD4+ T cells, effector memory CD4+ T cells, effector CD4+ T cells, CD4+ stem memory T cells, and bulk CD4+ T cells.

In some embodiments, the plurality of target lymphocytes includes CD8⁺ T cells, CD4⁺ T cells, gamma delta (γδ) T cells, natural killer (NK) cells, or a combination of any thereof. In some embodiments, the CD8⁺ T cell, CD4⁺ T cell, gamma delta T cell are PD1^{hi} cells. In some embodiments, the NK cells are CD56^{hi} and PD1^{hi} cells. In some embodiments, the NK cells are PD1^{lo} cells and CD16⁺ cells.

The cytokine molecule transports into each of the plurality of physiological compartments, and the plurality of target lymphocytes traffic to each of the plurality of physiological compartments, except for the lymph compartment 308. For example, in some embodiments, at least some or all of the plurality of physiological compartments includes the plurality of target lymphocytes. For example, the central compartment 302, the tight compartment 304, the leaky compartment 306, and the tumor compartment 308 may include each of the plurality of lymphocytes including the CD8+ T cell 312, the CD4+ T cell 314, the CD4−/CD8−(Double Negative) T cell 316, and the natural killer (NK) cell 318.

In some embodiments, each target lymphocyte of the plurality of target lymphocytes has dynamics parameters which govern baseline production, death, proliferation, and trafficking in and out of the plurality of physiological compartments. For example, the architecture 300 of the QSP model 1408 includes a dynamics parameter corresponding to the plurality of target lymphocytes within each of the plurality of physiological compartments. The dynamics parameter may include at least one of a quantity, a proliferation, an expansion, a contraction, a persistence, and a differentiation in each of the plurality of physiological compartments. The architecture 300 of the QSP model 1408 may additionally and/or alternatively include trafficking of the plurality of target lymphocytes between each of the plurality physiological compartments (except for the lymph compartment 308).

For example, the architecture 300 may include trafficking (e.g., via a trafficking rate) of the plurality of target lymphocytes between the central compartment 302 and the tight compartment 304, between the central compartment 302 and the leaky compartment 306, and between the central compartment 302 and the tumor compartment 308. Such embodiments may include trafficking (e.g., via a trafficking rate) of the plurality of target lymphocytes from the central compartment 302 to the tight compartment 304, from the tight compartment 304 to the central compartment 302, from the central compartment 302 to the leaky compartment 306, from the leaky compartment 306 to the central compartment 302, from the central compartment 302 to the tumor compartment 308, and/or from the tumor compartment 308 to the central compartment 302. In other embodiments, the architecture 300 includes trafficking of the plurality of target lymphocytes between the tight compartment 304, the leaky compartment 306, and the tumor compartment 308.

Referring again to FIG. 3A, the QSP model 1408 tracks IL-15 and PD1 receptor expression on the surface of each of the plurality of target lymphocytes in each of the corresponding physiological compartments. In some embodiments, IL-15 receptors (IL-15R) 320 bind to the cytokine molecule 202, 204 (e.g., Xmab24306 202 or the IL-15 arm of PD1/IL-15 TaCk 204). The PD1 arm of PD1/IL-15 TaCk 204 binds to PD1 receptors 322. The QSP model 1408 may specifically include avidity. Further, the QSP model 1408 may track the concentration of free receptor, receptors bound only to one arm of the cytokine molecule, and full drug: receptor complexes at the surface of every target lymphocyte in each corresponding physiological compartment.

For example, as shown in FIG. 3A, at least some or all of the plurality of physiological compartments includes the cytokine molecule. For example, the central compartment 302, the tight compartment 304, the leaky compartment 306, the tumor compartment 308, and the lymph compartment 308 may include the cytokine molecule (e.g., the PD1/IL15 TaCk 204, the XmAb-24306, and/or another cytokine molecule described herein). The cytokine molecule has a binding parameter which governs the behavior of the cytokine molecule within each of the plurality of physiological compartments. For example, the binding parameter includes at least one of an affinity and an avidity of the cytokine molecule within each of the plurality of physiological compartments.

In some embodiments, the binding parameter includes at least one of a concentration of free receptors at a surface of each of the plurality of target lymphocytes within each of the plurality of physiological compartments (e.g., the central compartment 302, the tight compartment 304, the leaky compartment 306, the tumor compartment 308, and the lymph compartment 310), a concentration of single bound drug: receptor complexes at the surface of each of the plurality of target lymphocytes within the central compartment 302, the tight compartment 304, the leaky compartment 306, and/or the tumor compartment 308, and a concentration fully bound cytokine molecule complexes at the surface of each of the plurality of target lymphocytes within the central compartment 302, the tight compartment 304, the leaky compartment 306, and/or the tumor compartment 308.

FIG. 3B schematically depicts an example 350 of the dynamics parameter and/or the binding parameter included in the architecture 300 of the QSP model 1408. For example, as noted the dynamics parameter may include at least one of a proliferation 352, a production 354, and a death rate 356 for each of the plurality of target lymphocytes (represented as the CD8+ T cell 312 in FIG. 3B). As noted, the architecture 300 may also include trafficking rate into (shown as 358) and/or out of (shown as 360) each corresponding one of the plurality of physiological compartments. Referring again to FIG. 3B, the architecture 300 may include a binding parameter, such as a synthesis 361 and a degradation 362 corresponding to the expression of the PD1 receptors 322 and/or the IL15R 320 at the surface of each of the target lymphocytes. This allows for tracking of the PD1 receptors 322 and the IL15R 320 at the surface of each of the target lymphocytes. Again referring to FIG. 3B, as noted above, the architecture 300 may specifically include a binding avidity 364 corresponding to the cytokine molecule and each of the PD1 receptors 322 and the IL15R 320 (e.g., at 366). While FIG. 3B shows one example of a target lymphocyte (e.g., the CD8+ T cell 312) and an example of the cytokine molecule (e.g., the PD1/IL15 TaCk cytokine molecule 204), the same binding parameters and/or dynamics parameters may be included in the architecture 300 for the CD4+ T cell 314, the CD4−/CD8−(Double Negative) T cell 316, and the natural killer (NK) cell 318, as well as the XmAb-24306 202 or other cytokine molecule.

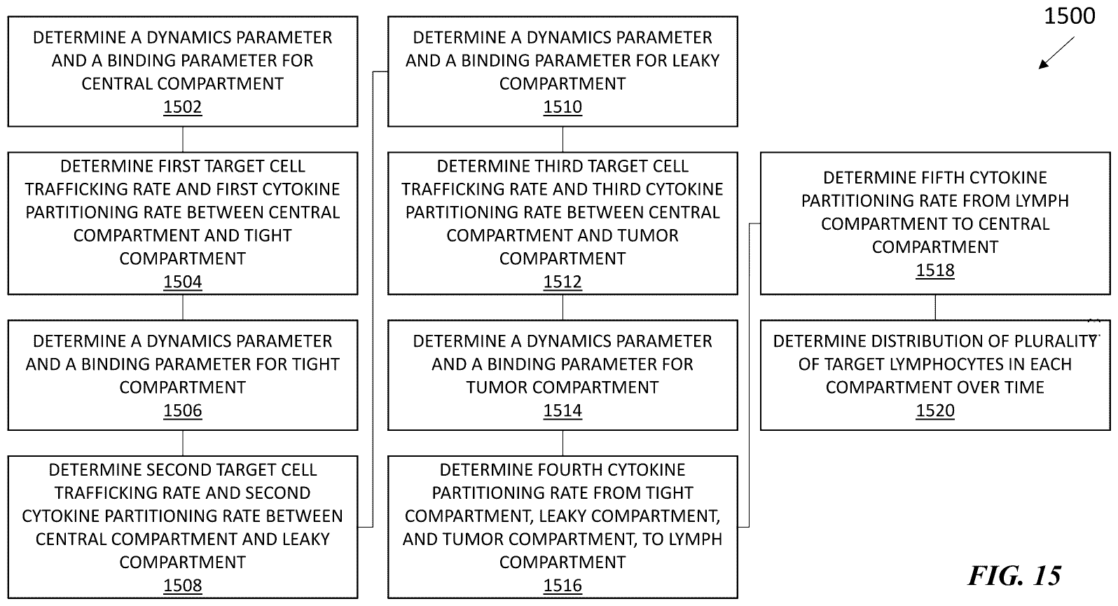

FIG. 15s depict a flowchart illustrating an example of a process 1500 for determining a distribution of a plurality of target lymphocytes over time after delivery of a cytokine molecule, consistent with implementations of the current subject matter. Referring to FIG. 15, the process 1500 may be performed by the analysis engine 1402 to generate a treatment plan, determine a cancer immunotherapy including a dose of a cytokine molecule, predict a patient response to a cytokine molecule, and/or the like. Consistent with implementations of the current subject matter, the process 1500 refers to the architecture 300 of the QSP model 1408 shown in FIGS. 3A and 3B.

At 1502, the analysis engine 1402 (e.g., at least one data processor) may determine a dynamics parameter and a binding parameter for the central compartment 302. For example, the analysis engine 1402 may determine a dynamics parameter corresponding to a plurality of target lymphocytes within a central compartment (e.g., the central compartment 302) of a patient. The analysis engine 1402 may also determine a binding parameter between the cytokine molecule (e.g., the PD1/IL15 TaCk 204, the XmAb-24306 202, and/or the like) and the plurality of target lymphocytes within the central compartment 302. The plurality of target lymphocytes include a CD8+ T cell, a CD4+ T cell, a CD4−/CD8−(Double Negative) T cell, and a natural killer (NK) cell. Each of the plurality of target lymphocytes may include a first subpopulation associated with high PD1 expression and a second subpopulation associated with low PD1 expression.

As noted, the cytokine molecule may include interleukin 2 (IL-2), interleukin 7 (IL-7), interleukin 12 (IL-12), interleukin 15 (IL-15), interleukin 21 (IL-21), or interferon. For example, the cytokine molecule may include the PD1/IL15 TaCk 204, the XmAb-24306 202, RSV IL-15 TaCk, or another cytokine molecule that stimulates an immune system of the patient and/or proliferates target lymphocytes. Additionally and/or alternatively, the cytokine molecule includes an engineered cytokine, such as one that agonizes its cognate receptor. Additionally and/or alternatively, the cytokine molecule is a bispecific molecule including a first arm and a second arm. Interleukin 15 receptors (IL-15R) of each of the plurality of target lymphocytes bind to the first arm of the bispecific molecule, and PD1 receptors of each of the plurality of target lymphocytes bind to the second arm of the bispecific molecule. Additionally and/or alternatively, the cytokine molecule is a multivalent polypeptide including a first polypeptide region capable of binding to a first target (e.g., a first receptor, such as IL-15R, of the cytokine molecule), and a second polypeptide region capable of binding to a second target (e.g., a second receptor, such as a PD1 receptor, of the cytokine molecule) and operably linked to the first polypeptide region. In some embodiments, the cytokine is an immunostimulatory cytokine.

The dynamics parameter includes at least one of a quantity, a proliferation, an expansion, a contraction, a persistence, and a differentiation of the plurality of target lymphocytes within the central compartment 302. The dynamics parameter corresponding to the plurality of target lymphocytes within the central compartment 302 may be determined at a time point. The dynamics parameter corresponding to the plurality of target lymphocytes within the central compartment 302 may be determined at a plurality of time points (e.g., a first time point, a second time point, a third time point, and so on). Thus, the analysis engine 1402 may capture the behavior of the plurality of target lymphocytes within the central compartment 302 at various time points.

The binding parameter may include at least one of an affinity and an avidity. The binding parameter may include at least one of a concentration of free receptors at a surface of each of the plurality of target lymphocytes within the central compartment 302, a concentration of single bound drug: receptor complexes at the surface of each of the plurality of target lymphocytes within the central compartment 302, and a concentration of fully bound cytokine molecule complexes at the surface of each of the plurality of target lymphocytes within the central compartment 302. Additionally and/or alternatively, the binding parameter is associated with expression of at least one of an interleukin 15 (IL-15) receptor and a PD1 receptor on a surface of each of the plurality of target lymphocytes within the central compartment 302. The binding parameter between the cytokine molecule and the plurality of target lymphocytes within the central compartment 302 may be determined at a plurality of time points (e.g., a first time point, a second time point, a third time point, and so on). Thus, the analysis engine 1402 may capture the binding behavior of the cytokine molecule with the plurality of target lymphocytes within the central compartment 302 at various time points.

At 1504, the analysis engine 1402 (e.g., at least one data processor) may determine a first target cell trafficking rate and a first cytokine partitioning rate between the central compartment 302 and the tight compartment 304. For example, the analysis engine 1402 may determine a first target cell trafficking rate of the plurality of target lymphocytes between the central compartment 302 and a tight compartment 304 of the patient. The analysis engine 1402 may also determine a first cytokine partitioning rate of the cytokine molecule between the central compartment 302 and the tight compartment 304.

The first target cell trafficking rate may correspond to trafficking of the plurality of target lymphocytes at line 348, shown in the architecture 300 of FIG. 3A. The first cytokine partitioning rate may correspond to partitioning (e.g., transportation) of the cytokine molecule at line 340, shown in the architecture 300 of FIG. 3A.

At 1506, the analysis engine 1402 (e.g., at least one data processor) may determine a dynamics parameter and a binding parameter for the tight compartment 304. For example, the analysis engine 1402 may determine the dynamics parameter corresponding to the plurality of target lymphocytes within the tight compartment 304. The analysis engine 1402 may also determine the binding parameter between the cytokine molecule and the plurality of target lymphocytes within the tight compartment 304.

The dynamics parameter includes at least one of a quantity, a proliferation, an expansion, a contraction, a persistence, and a differentiation of the plurality of target lymphocytes within the tight compartment 304. The dynamics parameter corresponding to the plurality of target lymphocytes within the tight compartment 304 may be determined at a time point. The dynamics parameter corresponding to the plurality of target lymphocytes within the tight compartment 304 may be determined at a plurality of time points (e.g., a first time point, a second time point, a third time point, and so on). Thus, the analysis engine 1402 may capture the behavior of the plurality of target lymphocytes within the tight compartment 304 at various time points.

The binding parameter may include at least one of an affinity and an avidity. The binding parameter may include at least one of a concentration of free receptors at a surface of each of the plurality of target lymphocytes within the tight compartment 304, a concentration of single bound drug: receptor complexes at the surface of each of the plurality of target lymphocytes within the tight compartment 304, and a concentration of fully bound cytokine molecule complexes at the surface of each of the plurality of target lymphocytes within the tight compartment 304. Additionally and/or alternatively, the binding parameter is associated with expression of at least one of an interleukin 15 (IL-15) receptor and a PD1 receptor on a surface of each of the plurality of target lymphocytes within the tight compartment 304. The binding parameter between the cytokine molecule and the plurality of target lymphocytes within the tight compartment 304 may be determined at a plurality of time points (e.g., a first time point, a second time point, a third time point, and so on). Thus, the analysis engine 1402 may capture the binding behavior of the cytokine molecule with the plurality of target lymphocytes within the tight compartment 304 at various time points.

At 1508, the analysis engine 1402 (e.g., at least one data processor) may determine a second target cell trafficking rate and a second cytokine partitioning rate between the central compartment 302 and the leaky compartment 306. For example, the analysis engine 1402 may determine a second target cell trafficking rate of the plurality of target lymphocytes between the central compartment 302 and a leaky compartment 306 of the patient. The analysis engine 1402 may also determine a second cytokine partitioning rate of the cytokine molecule between the central compartment 302 and the leaky compartment 306.

The second target cell trafficking rate may correspond to trafficking of the plurality of target lymphocytes at line 349, shown in the architecture 300 of FIG. 3A. The second cytokine partitioning rate may correspond to partitioning (e.g., transportation) of the cytokine molecule at line 342, shown in the architecture 300 of FIG. 3A.

At 1510, the analysis engine 1402 (e.g., at least one data processor) may determine a dynamics parameter and a binding parameter for the leaky compartment 306. For example, the analysis engine 1402 may determine the dynamics parameter corresponding to the plurality of target lymphocytes within the leaky compartment 306. The analysis engine 1402 may also determine the binding parameter between the cytokine molecule and the plurality of target lymphocytes within the leaky compartment 306.

The dynamics parameter includes at least one of a quantity, a proliferation, an expansion, a contraction, a persistence, and a differentiation of the plurality of target lymphocytes within the leaky compartment 306. The dynamics parameter corresponding to the plurality of target lymphocytes within the leaky compartment 306 may be determined at a time point. The dynamics parameter corresponding to the plurality of target lymphocytes within the leaky compartment 306 may be determined at a plurality of time points (e.g., a first time point, a second time point, a third time point, and so on). Thus, the analysis engine 1402 may capture the behavior of the plurality of target lymphocytes within the leaky compartment 306 at various time points.

The binding parameter may include at least one of an affinity and an avidity. The binding parameter may include at least one of a concentration of free receptors at a surface of each of the plurality of target lymphocytes within the leaky compartment 306, a concentration of single bound drug: receptor complexes at the surface of each of the plurality of target lymphocytes within the leaky compartment 306, and a concentration of fully bound cytokine molecule complexes at the surface of each of the plurality of target lymphocytes within the leaky compartment 306. Additionally and/or alternatively, the binding parameter is associated with expression of at least one of an interleukin 15 (IL-15) receptor and a PD1 receptor on a surface of each of the plurality of target lymphocytes within the leaky compartment 306. The binding parameter between the cytokine molecule and the plurality of target lymphocytes within the leaky compartment 306 may be determined at a plurality of time points (e.g., a first time point, a second time point, a third time point, and so on). Thus, the analysis engine 1402 may capture the binding behavior of the cytokine molecule with the plurality of target lymphocytes within the leaky compartment 306 at various time points.

At 1512, the analysis engine 1402 (e.g., at least one data processor) may determine a third target cell trafficking rate and a third cytokine partitioning rate between the central compartment 302 and the tumor compartment 308. For example, the analysis engine 1402 may determine a third target cell trafficking rate of the plurality of target lymphocytes between the central compartment 302 and a tumor compartment 308 of the patient. The analysis engine 1402 may also determine a third cytokine partitioning rate of the cytokine molecule between the central compartment 302 and the tumor compartment 308.

The third target cell trafficking rate may correspond to trafficking of the plurality of target lymphocytes at line 347, shown in the architecture 300 of FIG. 3A. The third cytokine partitioning rate may correspond to partitioning (e.g., transportation) of the cytokine molecule at line 346, shown in the architecture 300 of FIG. 3A.

At 1514, the analysis engine 1402 (e.g., at least one data processor) may determine a dynamics parameter and a binding parameter for the tumor compartment 308. For example, the analysis engine 1402 may determine the dynamics parameter corresponding to the plurality of target lymphocytes within the tumor compartment 308. The analysis engine 1402 may also determine the binding parameter between the cytokine molecule and the plurality of target lymphocytes within the tumor compartment 308.

The dynamics parameter includes at least one of a quantity, a proliferation, an expansion, a contraction, a persistence, and a differentiation of the plurality of target lymphocytes within the tumor compartment 308. The dynamics parameter corresponding to the plurality of target lymphocytes within the tumor compartment 308 may be determined at a time point. The dynamics parameter corresponding to the plurality of target lymphocytes within the tumor compartment 308 may be determined at a plurality of time points (e.g., a first time point, a second time point, a third time point, and so on). Thus, the analysis engine 1402 may capture the behavior of the plurality of target lymphocytes within the tumor compartment 308 at various time points.

The binding parameter may include at least one of an affinity and an avidity. The binding parameter may include at least one of a concentration of free receptors at a surface of each of the plurality of target lymphocytes within the tumor compartment 308, a concentration of single bound drug: receptor complexes at the surface of each of the plurality of target lymphocytes within the tumor compartment 308, and a concentration of fully bound cytokine molecule complexes at the surface of each of the plurality of target lymphocytes within the tumor compartment 308. Additionally and/or alternatively, the binding parameter is associated with expression of at least one of an interleukin 15 (IL-15) receptor and a PD1 receptor on a surface of each of the plurality of target lymphocytes within the tumor compartment 308. The binding parameter between the cytokine molecule and the plurality of target lymphocytes within the tumor compartment 308 may be determined at a plurality of time points (e.g., a first time point, a second time point, a third time point, and so on). Thus, the analysis engine 1402 may capture the binding behavior of the cytokine molecule with the plurality of target lymphocytes within the tumor compartment 308 at various time points.

At 1516, the analysis engine 1402 (e.g., at least one data processor) may determine a fourth cytokine partitioning rate from the tight compartment 304, the leaky compartment 306, and the tumor compartment 308, to the lymph compartment 1516. For example, the analysis engine 1402 may determine a fourth cytokine partitioning rate of the cytokine molecule from the tight compartment 304, the leaky compartment 306, and/or the tumor compartment 308, to (e.g., between) the lymph compartment 308. The fourth cytokine partitioning rate may correspond to partitioning (e.g., transportation) of the cytokine molecule at line 345, shown in the architecture 300 of FIG. 3A.

At 1518, the analysis engine 1402 (e.g., at least one data processor) may determine a fifth cytokine partitioning rate from the lymph compartment 1516 to the central compartment 302. For example, the analysis engine 1402 may determine a fifth cytokine partitioning rate of the cytokine molecule from (e.g., between) the lymph compartment 308 and the central compartment 302. The fifth cytokine partitioning rate may correspond to partitioning (e.g., transportation) of the cytokine molecule at line 343, shown in the architecture 300 of FIG. 3A.

At 1520, the analysis engine 1402 (e.g., at least one data processor) may determine a distribution of the plurality of target lymphocytes in each compartment over time. For example, the analysis engine 1402 may determine a distribution of each of the plurality of target lymphocytes in the central compartment 302, the tight compartment 304, the leaky compartment 306, and the tumor compartment 308 over time. The analysis engine 1402 may determine the distribution based on the dynamics parameter, the binding parameter, the first target cell trafficking rate, the second target cell trafficking rate, the third target cell trafficking rate, the first cytokine partitioning rate, the second cytokine partitioning rate, the third cytokine partitioning rate, the fourth cytokine partitioning rate, and/or the fifth cytokine partitioning rate.

The distribution may include a first distribution corresponding to the cytokine molecule delivered to the patient at a first time point and a second distribution corresponding to the cytokine molecule delivered to the patient at a second time point, and so on. The analysis engine 1402 may determine a response of another patient (e.g., a second patient) to a cancer immunotherapy including the cytokine molecule, based at least on the first distribution and the second distribution. Additionally and/or alternatively, the analysis engine 1402 may determine a treatment plan for the second patient based on the response of the second patient to the cancer immunotherapy. Additionally and/or alternatively, the analysis engine 1402 may determine a cancer immunotherapy for treating a tumor. For example, the analysis engine 1402 may determine a dose of the cytokine molecule and a dosing frequency for delivering the dose of the cytokine molecule, based at least on the distribution.

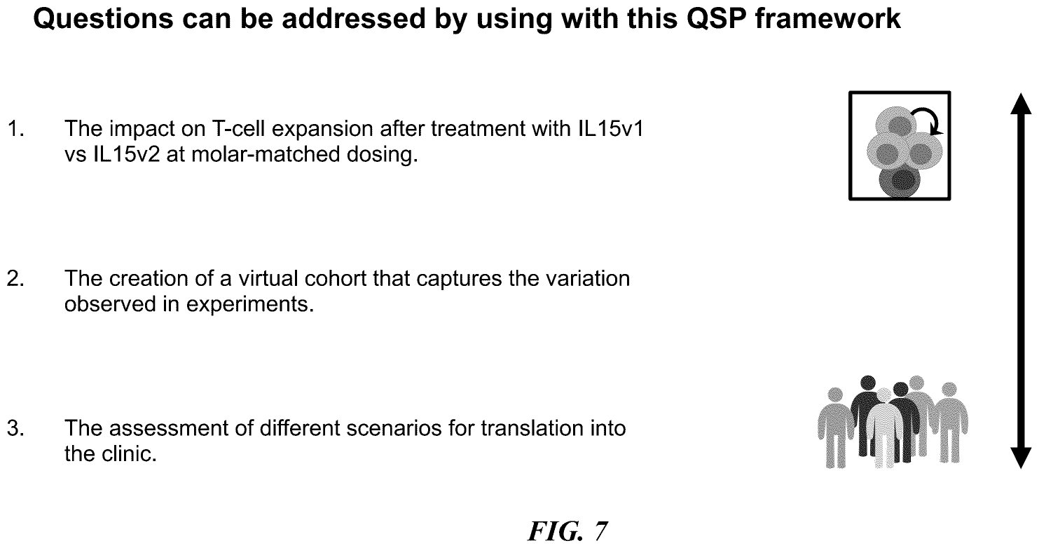

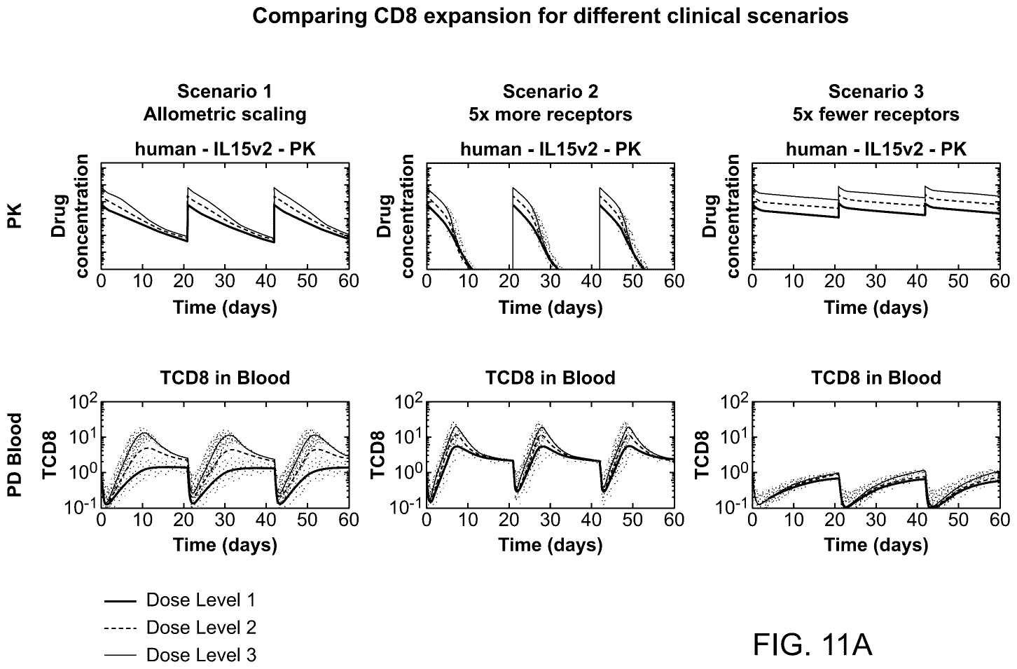

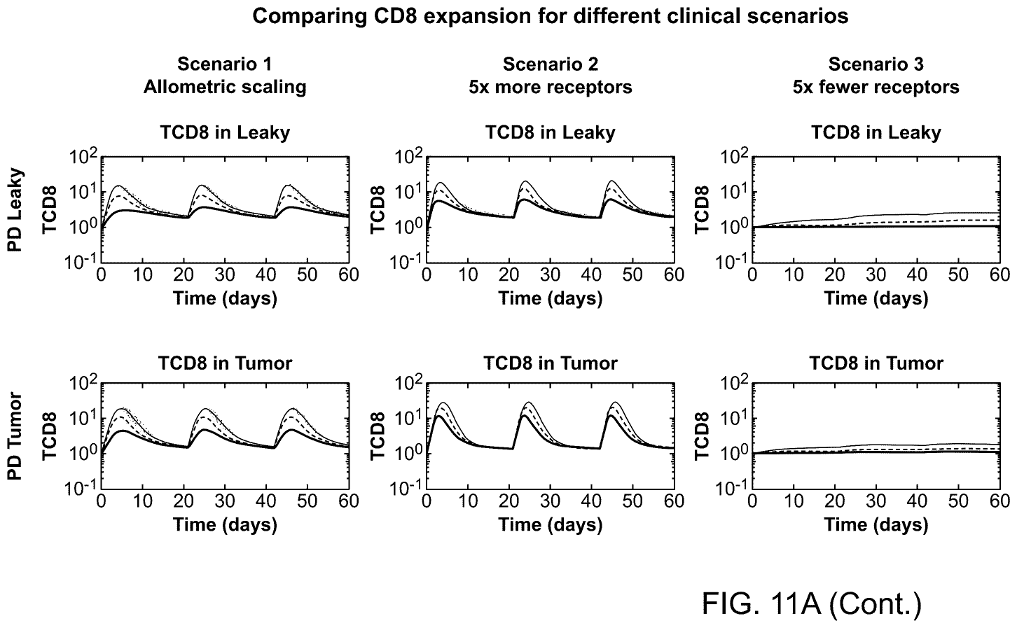

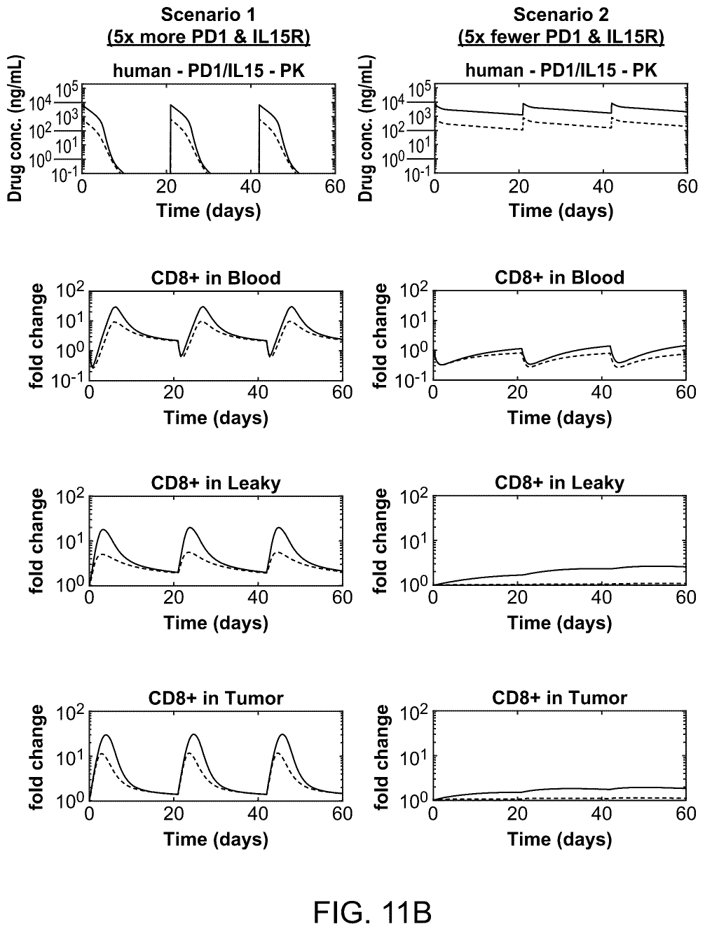

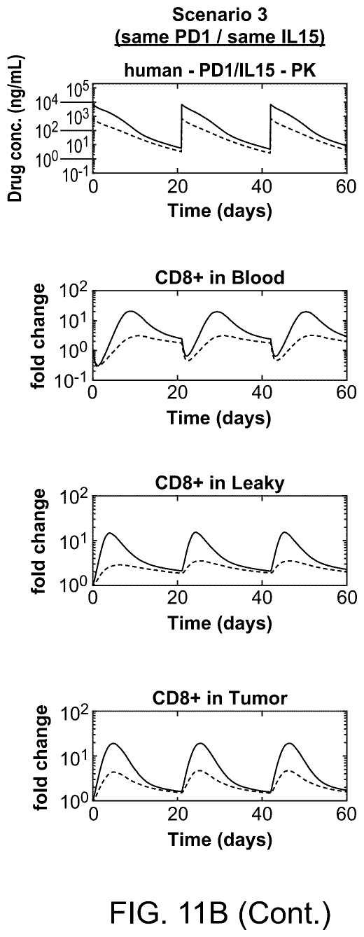

Thus, the analysis engine 1402 may employ the QSP model 1408 to determine an impact on T-cell and/or target lymphocyte expansion after treatment with at least one cytokine molecule, create a virtual cohort that captures variations of cytokine molecules, and/or assess various scenarios for translation to a clinical setting (see FIG. 7). The analysis engine 1402 may additionally and/or alternatively balance lymphocyte expansion in different tissues versus tumors based on the target expression, which could be beneficial in the early stages of drug development. Additionally and/or alternatively, the analysis engine 1402 supports preclinical, translational, and early clinical development of cytokine molecules, such as engineered cytokines that act to activate the immune system and expand target cells for cancer immunotherapy. The analysis engine 1402 also allows for the determination of the impact on a change in one or more parameters (e.g., the dynamics parameter and/or the binding parameter), one or more doses, and/or one or more lymphocytes. For example, FIG. 11A and FIG. 11B depict comparisons of CD8 expansion for different clinical scenarios. As shown in FIG. 11A and FIG. 11B, for example, a five-fold increase in receptors (e.g., PD1 and IL15R) has a slightly higher expansion at lower doses, and a five-fold decrease in receptors (e.g., PD1 and IL15R) causes mass reduction in expansion. As another example, as shown in FIG. 11B,

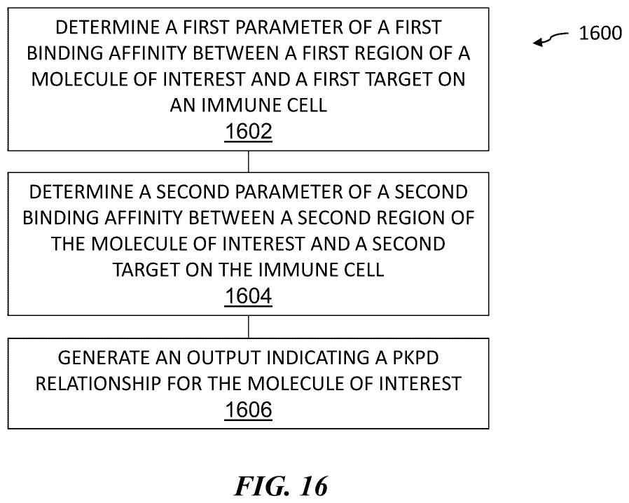

FIG. 16 depicts a flowchart illustrating an example of a process 1400 for quantitative systems pharmacology (QSP) modeling. In some example embodiments, the analysis engine 1402 may perform the process 1400 to model the mechanisms of drug pharmacokinetics, pharmacodynamics, dynamic target-mediated drug disposition, cellular expansion, cellular proliferation, and target receptor expression in a plurality of tissues. In some embodiments, the analysis engine 1402 may determine parameters having a binding affinity with the molecule of interest and indicate whether a PKPD relationship for the molecule of interest exists.

At 1602, the analysis engine 1402 may determine a first parameter of a first binding affinity between a first region of a molecule of interest and a first target on an immune cell. For example, the analysis engine 1402 may determine that a polypeptide of an engineered cytokine and a IL-15R of a CD8+ T cell has the first parameter of a first binding affinity. Examples of immune cells include CD8+ T cell, CD4+ T cell, gamma delta T cell (i.e., double negative T cell) and natural killer (NK) cell. Examples of molecules of interest include an engineered cytokine or a polypeptide with two regions that independently bind to two targets. The first target may be may be IL15R and the first region may be a polypeptide capable of binding to IL-15R (i.e., CD122 receptor). In some example embodiments, the analysis engine 1402 may be configured to determine a first parameter of a first binding affinity between a first region of an IL-15 agonist of interest and a first target on an immune cell.

At 1604, the analysis engine 1402 may determine a second parameter of a second binding affinity between a second region of the molecule of interest and a second target on the immune cell. The first binding affinity and the second binding affinity may be measured in multiple compartments. For example, the analysis engine 1402 may measure the first binding affinity in a tight compartment and the analysis engine 1402 may measure the second binding affinity in a lymph compartment. Examples of compartments include a tight compartment, a tumor compartment, a lymph compartment, and a central compartment. In some embodiments, the analysis engine 1402 may determine a polypeptide capable of binding to PD1 of the IL-15 agonist of interest and a PD1 on the CD4+ T cell has the second parameter of a second binding affinity. Examples of second regions include a polypeptide capable of binding to PD1. Examples of the second target may be PD1. In some embodiments, the analysis engine 1402 may determine a second parameter of a second binding affinity between a second region of the IL-15 agonist of interest and a second target on the immune cell.

At 1606, the analysis engine 1402 may generate an output indicating a PKPD relationship for the molecule of interest. The output indicating the PKPD relationship may be based on at least the first parameter and the second parameter. For example, the analysis engine 1402 may generate an output indicative of a PKPD relationship in which the binding of the second region to the second target has a higher K_{d} of 2nd interaction when the first region binds to the first target. In some embodiments, the analysis engine 1402 may generate an output indicating a PKPD relationship for the IL-15 agonist of interest based on the first parameter and the second parameter.

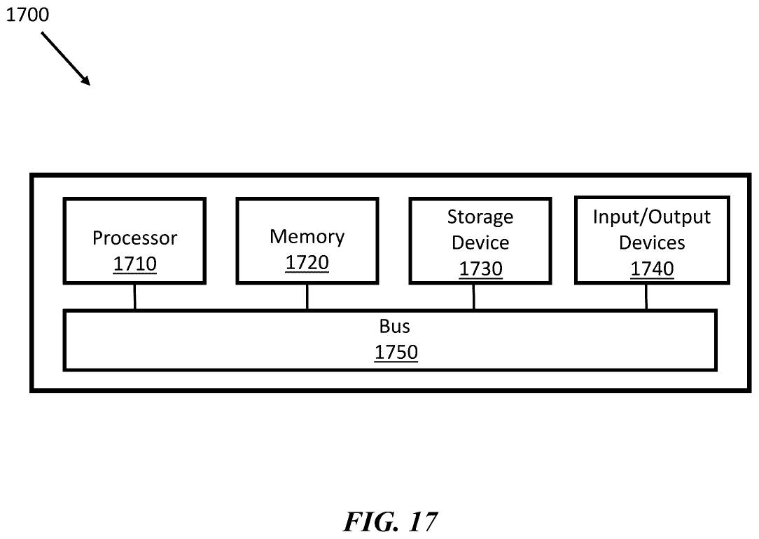

FIG. 17 depicts a block diagram illustrating an example of a computing system 1500 consistent with embodiments of the current subject matter. Referring to FIGS. 1-16, the computing system 1500 may be used to implement the analysis engine 1402 and/or any component therein. For example, the computing system 1500 may implement a user equipment, a personal computer, or a mobile device.

As shown in FIG. 17, the computing system 1700 may include a processor 1710, a memory 1720, a storage device 1730, and an input/output device 1740. The processor 1710, the memory 1720, the storage device 1730, and the input/output device 1740 may be interconnected via a system bus 1750. The processor 1710 is capable of processing instructions for execution within the computing system 1700. Such executed instructions may implement one or more components of, for example, the analysis engine 1402 for performing a quantitative systems pharmacology (QSP) modeling. In some example embodiments, the processor 1710 may be a single-threaded processor. Alternately, the processor 1710 may be a multi-threaded processor. The processor 1710 is capable of processing instructions stored in the memory 1720 and/or on the storage device 1730 to display graphical information for a user interface provided via the input/output device 1740.

The memory 1720 is a non-transitory computer-readable medium that stores information within the computing system 1700. The memory 1720 may store data structures representing configuration object databases, for example. The storage device 1730 is capable of providing persistent storage for the computing system 1700. The storage device 1730 may be a floppy disk device, a hard disk device, an optical disk device, or a tape device, or other suitable persistent storage means. The input/output device 1740 provides input/output operations for the computing system 1700. In some example embodiments, the input/output device 1740 includes a keyboard and/or pointing device. In various embodiments, the input/output device 1740 includes a display unit for displaying graphical user interfaces.

According to some example embodiments, the input/output device 1740 may provide input/output operations for a network device. For example, the input/output device 1740 may include Ethernet ports or other networking ports to communicate with one or more wired and/or wireless networks (e.g., a local area network (LAN), a wide area network (WAN), the Internet, a public land mobile network (PLMN), and/or the like).

In some example embodiments, the computing system 1700 may be used to execute various interactive computer software applications that may be used for organization, analysis, and/or storage of data in various formats. Alternatively, the computing system 1700 may be used to execute any type of software applications. These applications may be used to perform various functionalities, e.g., planning functionalities (e.g., generating, managing, editing of spreadsheet documents, word processing documents, and/or any other objects, etc.), computing functionalities, communications functionalities, etc. The applications may include various add-in functionalities or may be standalone computing items and/or functionalities. Upon activation within the applications, the functionalities may be used to generate the user interface provided via the input/output device 1740. The user interface may be generated and presented to a user by the computing system 1700 (e.g., on a computer screen monitor, etc.).

All publications and patent applications mentioned in this disclosure are herein incorporated by reference to the same extent as if each individual publication or patent application was specifically and individually indicated to be incorporated by reference.

No admission is made that any reference cited herein constitutes prior art. The discussion of the references states what their authors assert, and the inventors reserve the right to challenge the accuracy and pertinence of the cited documents. It will be clearly understood that, although a number of information sources, including scientific journal articles, patent documents, and textbooks, are referred to herein; this reference does not constitute an admission that any of these documents forms part of the common general knowledge in the art.

The discussion of the general methods given herein is intended for illustrative purposes only. Other alternative methods and alternatives will be apparent to those of skill in the art upon review of this disclosure, and are to be included within the spirit and purview of this application.

### EXAMPLES

Additional embodiments are disclosed in further detail in the following examples, which are provided by way of illustration and are not in any way intended to limit the scope of this disclosure or the claims.

The Examples below describe experiments performed to develop a QSP model (e.g., the QSP model 1408 including the architecture 300) that captures the complex interactions between drug pharmacokinetics (PK) and the differential expansion of multiple immune cell subsets across tissues and tumor.

A quantitative systems pharmacology (QSP) model (e.g., the architecture 300 of the QSP model 1408) was developed based on a minimal application of physiological based pharmacokinetic (PBPK) framework including the central compartment 302, the leaky compartment 306, the tight compartment 304, and the tumor compartment 308, to capture the biodistribution of the cytokine molecule (see, e.g., FIG. 3A and FIG. 3B). Two subtypes of PD1+ and PD1-cells were included for NK cells as well as CD4+, CD8+ and gamma delta T-cells in each compartment of the model. IL-15R (representing CD122/CD132 complex) and PD1 associated with each cell subtype was included in each compartment. The model takes into account the formation of single arm complexes as well as the avidity to form the triple complex (see FIG. 3B). As described in greater detail below, variants were defined to represent the properties of the three molecules used to calibrate the model: 1) XmAb24306, and 2) lead PD1/IL-15 TaCk molecule (see, e.g., FIG. 2).

As described herein, XmAb24306 is a fused IL-15/IL-15RA on half-life extended Fc domain and was developed with reduced potency to improve tolerability of the molecule. The molecule is currently being tested in clinic as a single agent or in combination with one or more additional treatments in patients with solid tumors. A PD1-targeted candidate (PD1/IL-15 TaCk) with a similar IL-15 targeting arm was also developed to promote selective expansion of PD1+ cell populations. The goal of the model developed herein is to better understand the pharmacokinetics (PK) of PD1/IL-15 TaCk due to target-mediated drug disposition (TMDD) and characterize the resulting cell expansion that could differentiate PD1/IL-15 TaCk from XmAb24306.

The model assumes that the number of bound IL-15Rs per cell drives the proliferation of different cell types. The baseline numbers of cell types were determined abased on existing data. The QSP model 1408 was calibrated to the in vitro potency data as well as in vivo data in cynomolgus monkey for both molecules and was translated to human through allometric scaling of the clearance and translation of receptor expression, cell numbers and physiological volumes.

i. Xmab24306, PD1 IL-15 TaCk, and RSV IL-15 TaCk Molecules

Three exemplary molecules that could act as agonists for the IL-15 receptor have been constructed. As described herein, the first molecule, Xmab24306, is a single-armed, engineered IL-15/IL-15Rα complex engineered using a heterodimeric Fc domain and Xtend™ half-life extension mutations. By complexing IL-15 and IL-15Rα (CD215) on the same Fc domain, Xmab24306 selectively engages IL2Rβ (CD122) and the common gamma-chain (γ) receptor (CD132) without engaging IL2Rα (CD25). The second molecule is PD1/IL-15 Targeted-Cytokine (TaCk), an anti-programmed cell death protein 1/interleukin-15 targeted two-armed engineered cytokine that selectively delivers IL-15 to cells that express PD1 with the goal of increasing T cell activation and proliferation. The third molecule, RSV/IL-15 TaCk, is similar to PD1/IL-15 but lacks the PD1 binding arm and assisted in model calibration. All molecules have been engineered to have extended half-lives compared to their endogenous IL-15 analogue, in an effort to increase PK exposure across time.

ii. OSP Model Calibration: Identifying a “Reference Virtual Cyno”

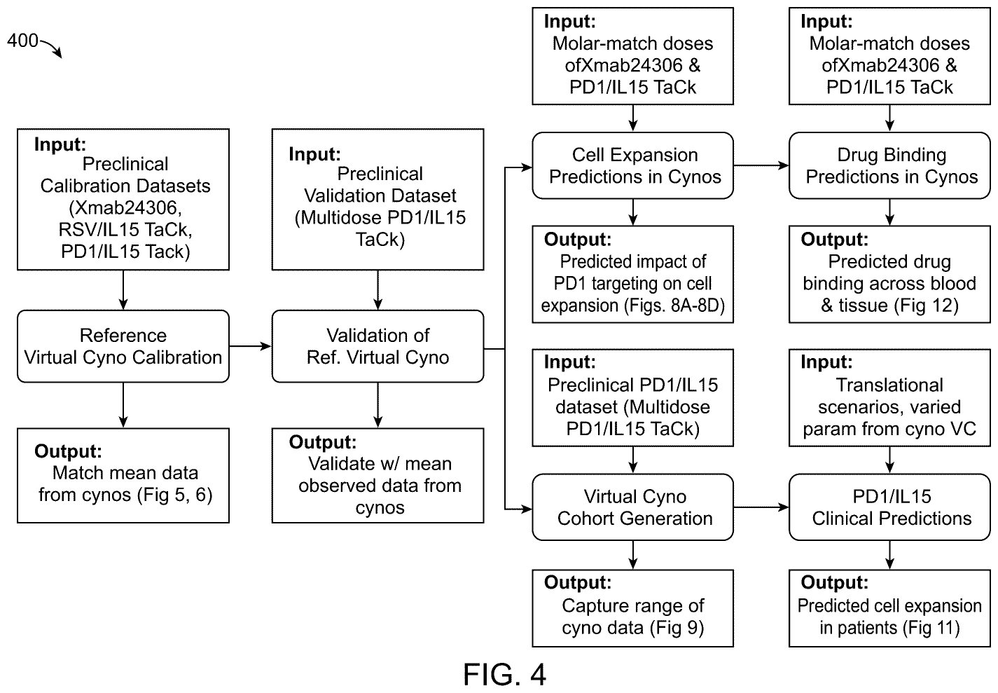

The QSP model (e.g., the QSP model 1408) was simultaneously calibrated using workflow 400 (see FIG. 4) to multiple distinct datasets including PK and PD in cynomolgus macaques (cynos) across multiple dose levels for the three engineered IL-15 cytokines, including Xmab24306, RSV/IL-15 TaCk, and PD1/IL-15 TaCk (see, e.g., FIG. 4). The QSP model was calibrated using global optimization methods, and the development of virtual subjects to explore the relationship between model parameters and model outcomes. The outcome is a calibrated reference virtual cyno, which reproduces the dynamics of lymphocyte expansion as well as PK profiles following treatment with one of the three molecules. The model was then validated by using the reference virtual cyno to predict the PK and lymphocyte expansion profiles following multi-dosing with PD1/IL-15 TaCk. Next, the reference virtual cyno was translated to a reference virtual patient by allometrically scaling physiological parameters and calibrating receptor turnover, receptor expression, and cellular proliferation to Xmab24306 phase 1a clinical trial data. To capture observed variability in cyno experimental measurements, a virtual cohort was generated that reasonably matched the observed range of measurements in the validation PD1/IL-15 TaCk dataset. The reference virtual patient was used together with the variability in the virtual cyno cohort to inform translational first-in-human dose predictions of PD1/IL-15 TaCk in patients.

Successful model calibration is defined as the identification of a single architecture (e.g., architecture 300) and associated parameter estimates (e.g., the dynamics parameters and/or the binding parameters) that reasonably describe both the PK and PD in cynos following treatment with any of the three molecules. A “reference virtual cyno” is defined as that accurately characterizes the PK and PD of each molecule in cynos.

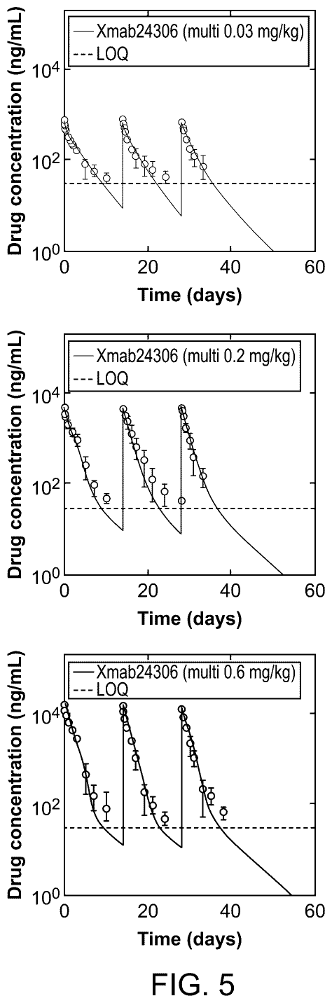

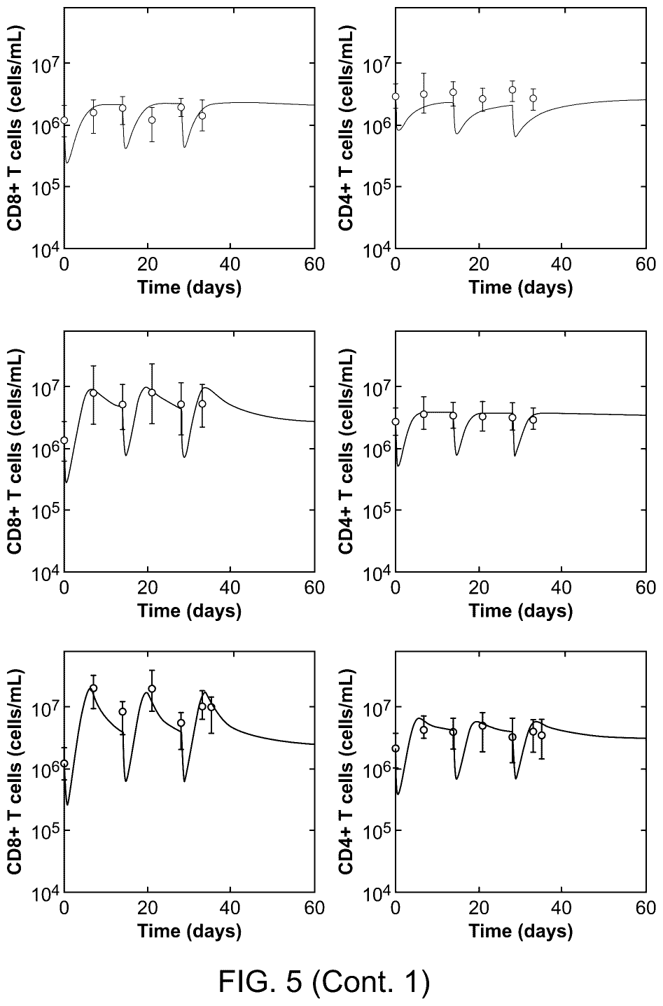

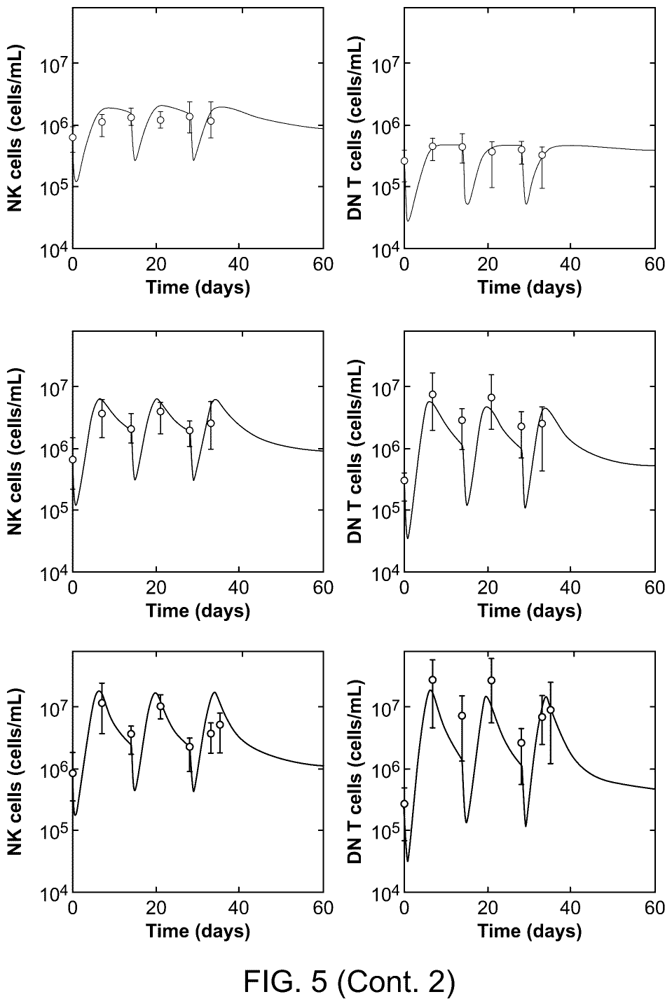

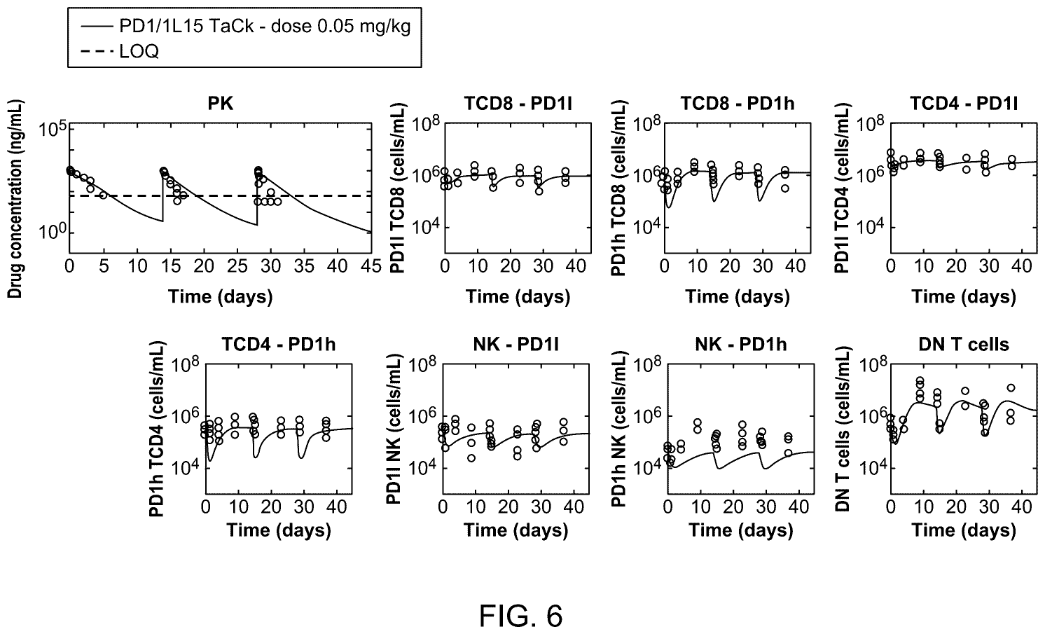

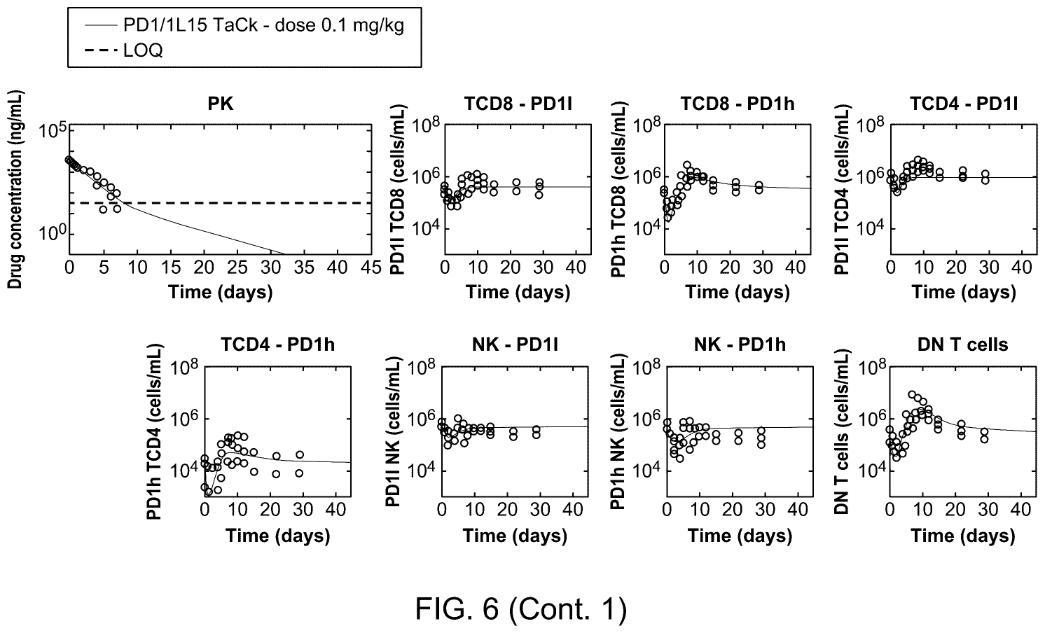

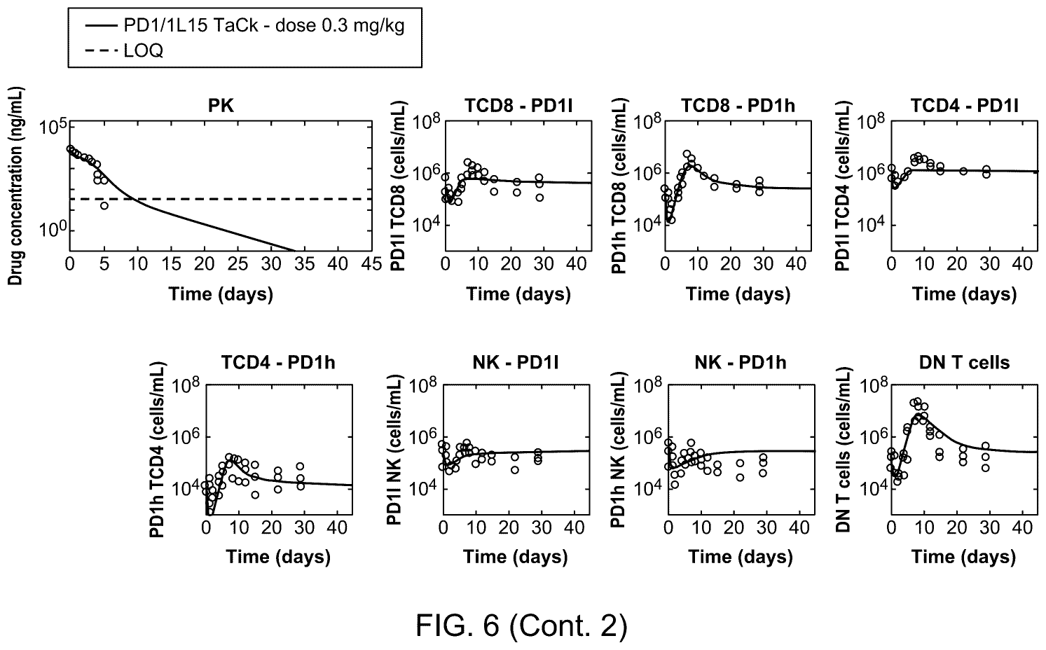

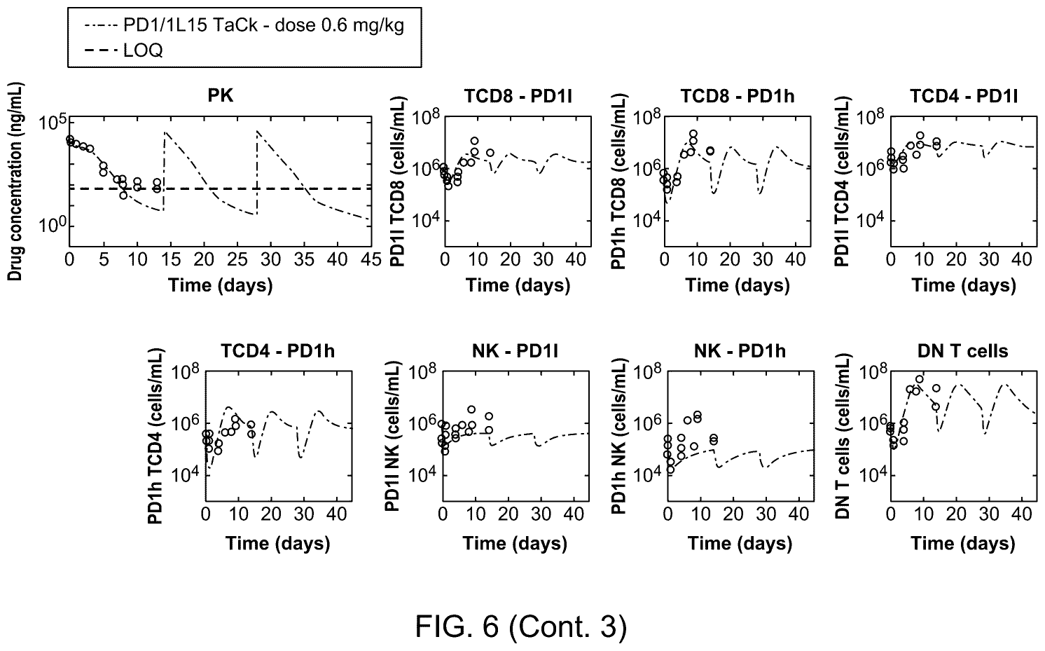

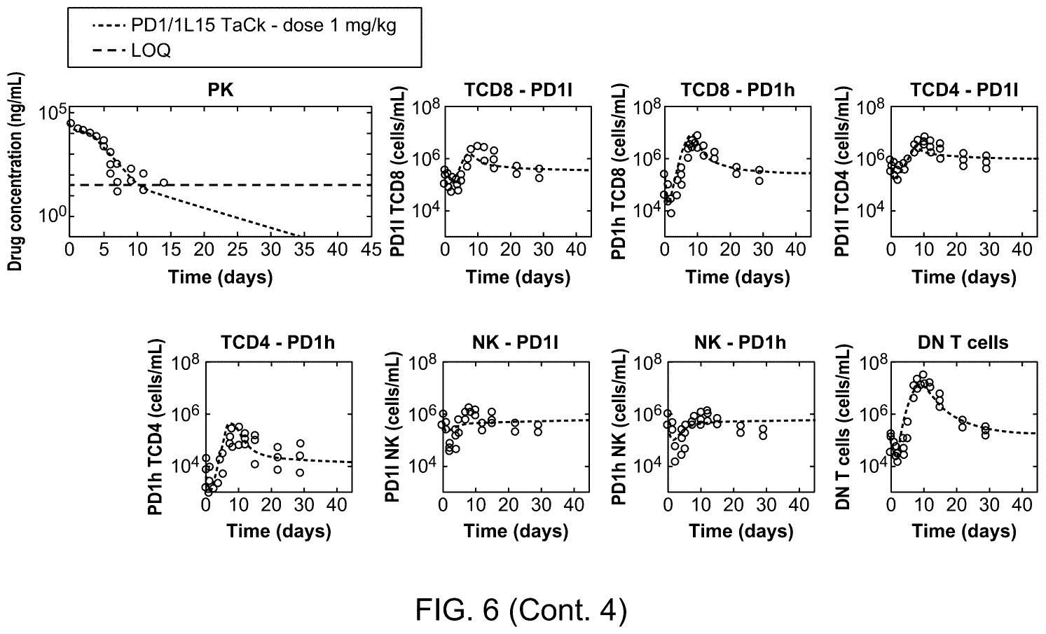

FIGS. 5 and 6 depict an exemplary calibrated QSP model (e.g., the calibrated QSP model 1408) that captures the dynamic relationship between PK and lymphocyte expansion. The reference virtual cyno model replicates lymphocyte expansion and PK profiles in the blood following treatment with A) Xmab24306 and B) PD1/IL-15 TaCk. Each row in FIGS. 5 and 6 shows a different dose level, ranging from 0.03 to 0.6 mg/kg for Xmab24306 (FIG. 5) and ranging from 0.06 to 1 mg/kg for PD1/IL-15 TaCk (FIG. 6). Each column shows experimental measurements in blood: either (A) total CD8+ T cells, CD4+ T cells, NK cells, double-negative T cells or (B) PD1^{lo} (PD1l) and PD1^{hi} (PD1h) subpopulations for all cells except double-negative T cells.

For example, referring to FIG. 5, the first column depicts plots showing drug concentration levels over time following delivery of multiple doses of 0.03 mg/kg of Xmab24306 (e.g., IL15v1) in the first row, 0.2 mg/kg of Xmab24306 (e.g., IL15v1) in the second row, and 0.6 mg/kg of Xmab24306 (e.g., IL15v1) in the third row. The second column depicts a concentration of CD8+ T cells over time following delivery of the corresponding doses. The third column depicts a concentration of CD4+ T cells over time following delivery of the corresponding doses. The fourth column depicts a concentration of NK cells over time following delivery of the corresponding doses. The fifth column depicts a concentration of double negative T cells over time following delivery of the corresponding doses.

Referring to FIG. 6, first column depicts plots showing drug concentration levels over time following delivery of a dose of 0.05 mg/kg of PD1/IL15 TaCk in the first row, 0.1 mg/kg of PD1/IL15 TaCk in the second row, 0.3 mg/kg of PD1/IL15 TaCk in the third row, and 0.6 mg/kg of PD1/IL15 TaCk in the fourth row. The second column depicts a concentration of CD8+T PD1^{hi} cells over time following delivery of the corresponding dose. The third column depicts a concentration of CD8+PD1^{lo} T cells over time following delivery of the corresponding dose. The fourth column depicts a concentration of CD4+PD1^{hi} T cells over time following delivery of the corresponding dose. The fifth column depicts a concentration of CD4+PD1^{lo} Tcells over time following delivery of the corresponding dose. The sixth column depicts a concentration of NK PD1^{hi} T cells over time following delivery of the corresponding dose. The seventh column depicts a concentration of NK PD1^{lo} T cells over time following delivery of the corresponding dose. The eighth column depicts a concentration of double negative T cells over time following delivery of the corresponding dose. The QSP model is a fully coupled PKPD model in which the pool of targets (PD1 and IL-15R) is significantly impacted by PK through cell proliferation and this cellular expansion subsequently affects PK through TMDD. The model successfully reproduces the impact of dynamic TMDD on PK and predicts that the lymphocytes in the tissues rather than blood and tumor are likely the main driver of TMDD due to 50× higher absolute T-cell counts in the tissues. Additionally, assuming PD1 expression is similar across tissues, the model predicts that the expansion in the circulation and tumor is proportional to the expansion in healthy tissues. However, cellular expansion is primarily driven by PD1^{hi} cells following treatment with PD1/IL15 TaCk, whereas treatment with Xmab24306 similarly expands both PD1^{lo} and PD1^{hi} cells in the tissue.

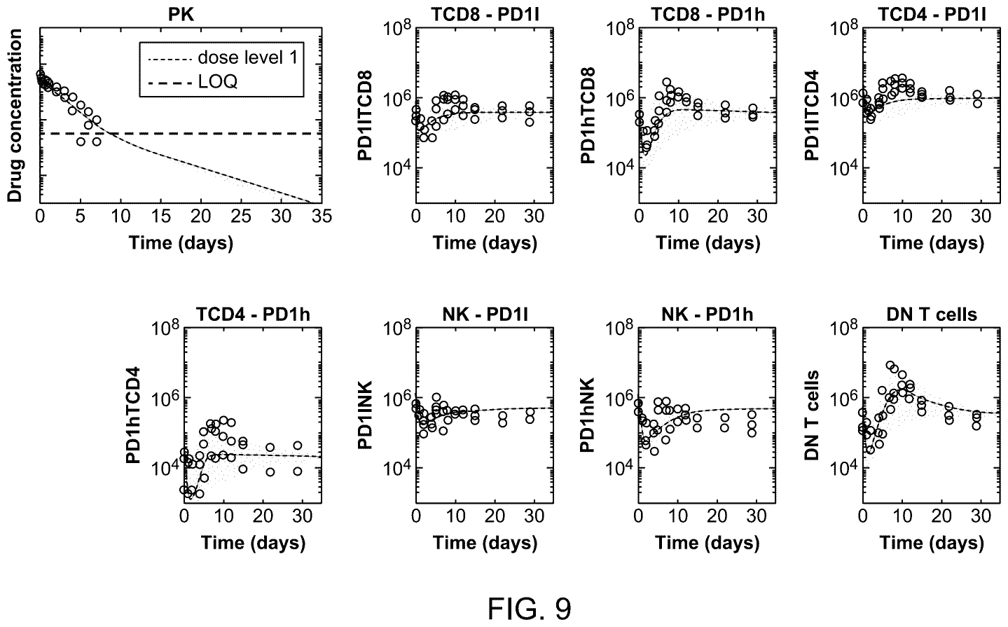

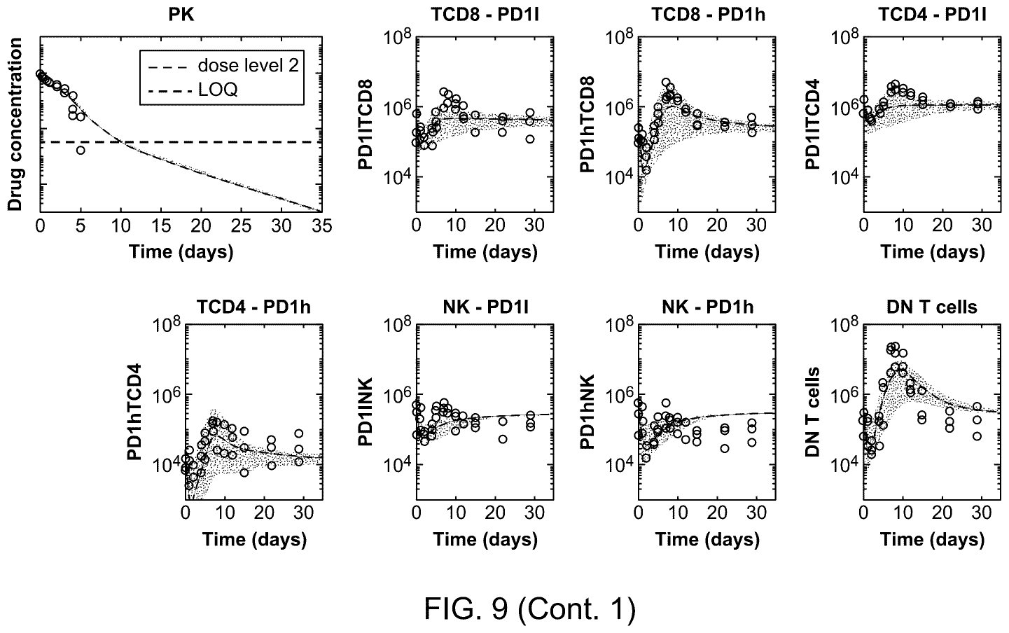

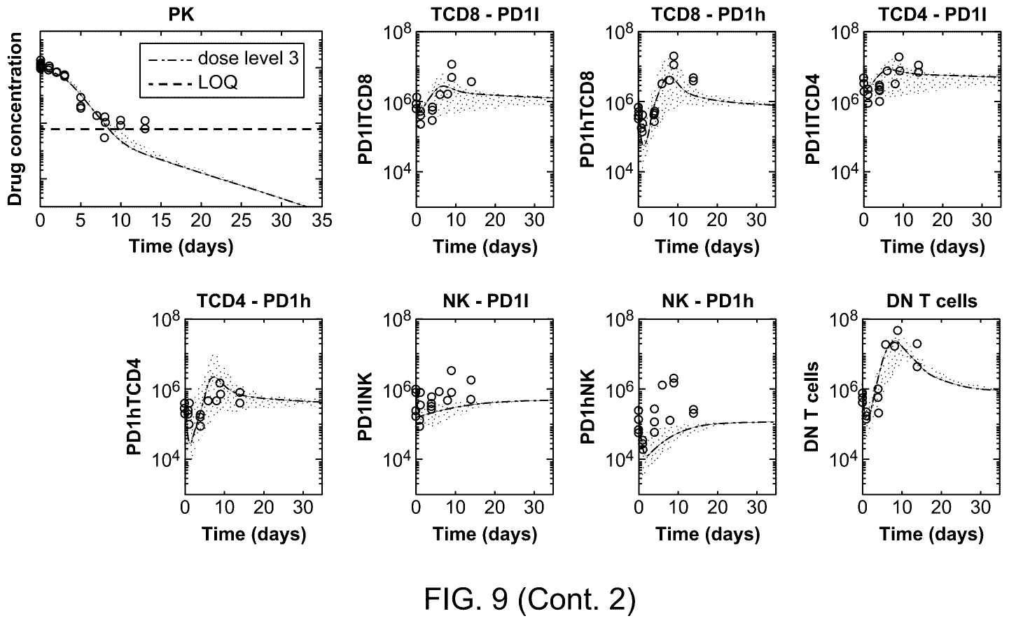

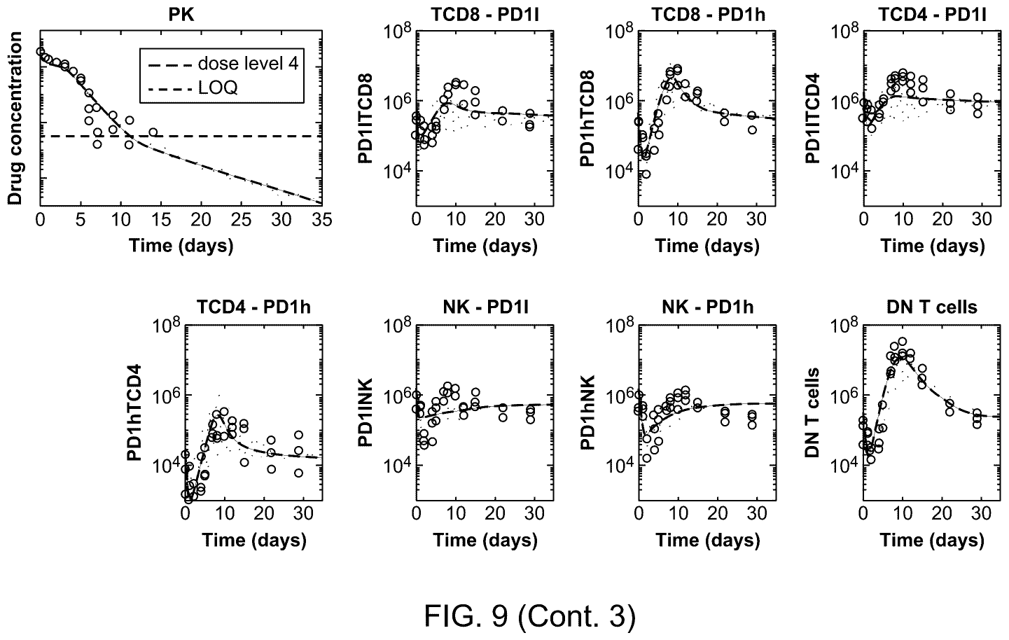

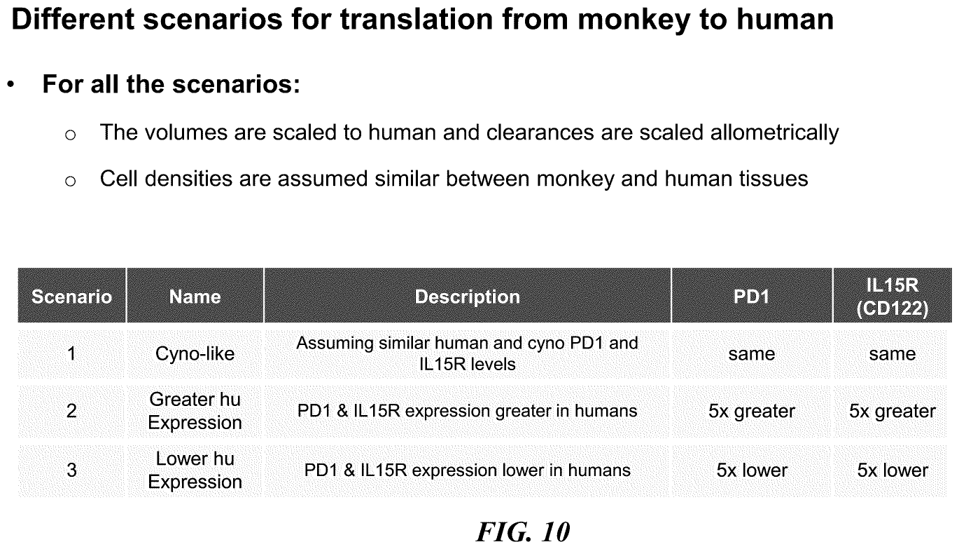

Generally, model parameter values may be altered to capture the variability of response illustrated in experimental data (see, e.g., FIG. 9), but exploration of parameter space was also performed to make predictions about the differential human and monkey response to IL-15 targeting molecules. Furthermore, the translated human model, based on three different scenarios (see, e.g., FIG. 10), predicted that less cellular expansion in the blood is to be expected in patients compared to cynomolgus monkeys and that cellular expansion across tissues and tumor is sensitive to target receptor expression.

i. Reference Virtual Cyno Captures Response of Multiple Cell Types to Different Engineered IL-15 Cytokines at Different Dose Levels

After model construction and calibration, the reference virtual cyno recapitulates lymphocyte expansion following treatment with any of three engineered IL-15 cytokines. Moreover, the model development and calibration processes disclosed herein enabled delineating key mechanisms that drive the dynamics between PK and PD across the IL-15 engineered cytokines. For example, the same reference virtual cyno parameter set captures both the robust, dose-dependent NK cell response induced by Xmab24306 while simultaneously capturing the lack of response in NK cells following treatment with PD1/IL-15 TaCk at any dose level (see, e.g., FIGS. 5-6). In lieu of an NK-driven response, the simulation described herein could recapitulate the CD8+, CD4+ and DN T cell response that is characteristic of treatment with PD1/IL-15 TaCk.

The reference virtual cyno also captures the temporal dynamics present within the preclinical data for each engineered cytokine. Upon first exposure to Xmab24306 (see, e.g., FIG. 5 or PD1/IL-15 TaCk (see, e.g., FIG. 6), drug-induced stimulation increases margination and trafficking of lymphocytes from the blood, leading to a rapid drop in cellular counts in the blood, consistent with other treatments. Drug partitioning to tissues leads to local lymphocyte activation, expansion and proliferation, causing a dose-dependent expansion of tissue cells that eventually re-enter to the blood stream approximately 7 to 14 days following dosing, depending on molecule and cell type (see, e.g., FIGS. 5-6). As drug concentrations decline, cells eventually die and return to steady state levels. These dynamics are captured by the newly developed reference virtual cyno across multiple dosages and formulations of engineered cytokines. The reference virtual cyno was used as a starting point for subsequent translational and cohort generation efforts.

ii. OSP Model Captures Dynamics of Validation Dataset

Next, the predictions obtained with the calibrated model (e.g., the calibrated QSP model 1408) were validated as follows. PD1/IL-15 TaCk treatment was simulated for the newly developed reference virtual cyno across four separate dose levels—a control scenario (vehicle, no drug), and treatment at 0.03, 0.1 and 0.3 mg/kg (Q3W dosing). Predicted PK and lymphocyte expansion were compared to a separate preclinical dataset that was not used to calibrate the model. For the majority of cells across each dose level, the disclosed model predictions recapitulated key dynamics of the experimental dataset, namely the margination and subsequent peak in blood cell counts following each dosing event. The agreement between model predictions and experimental measurements provides confidence in the ability of the model to predict lymphocyte dynamics following treatment to an alternate dosing regimen (3 doses) and doses as low as 0.03 mg/kg. Model predictions for the control case align well with experimental data. The validation of the model structure and reference virtual cyno parameter values described herein provide confidence in the model predictions.

iii. Comparison of Xmab24306 and PD IL-15 TaCk Simulations Illustrates Impact of PD1-Targeting During Treatment

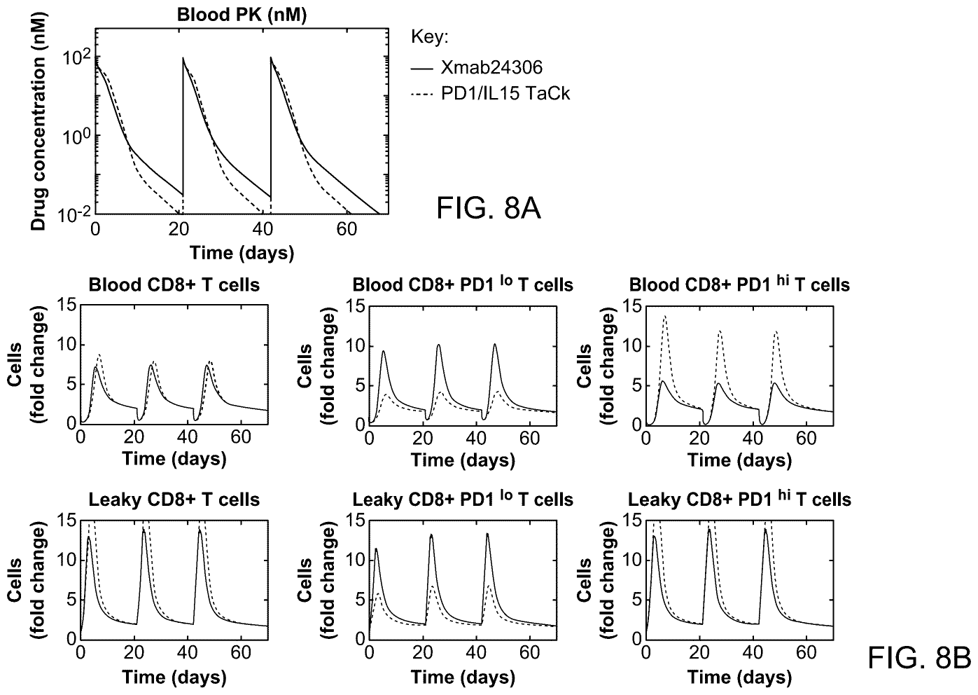

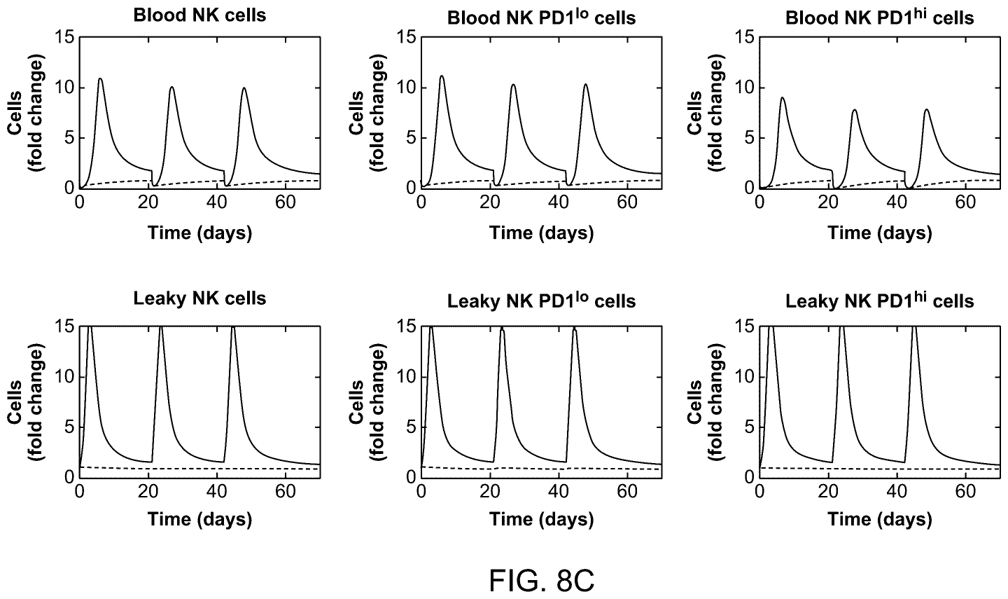

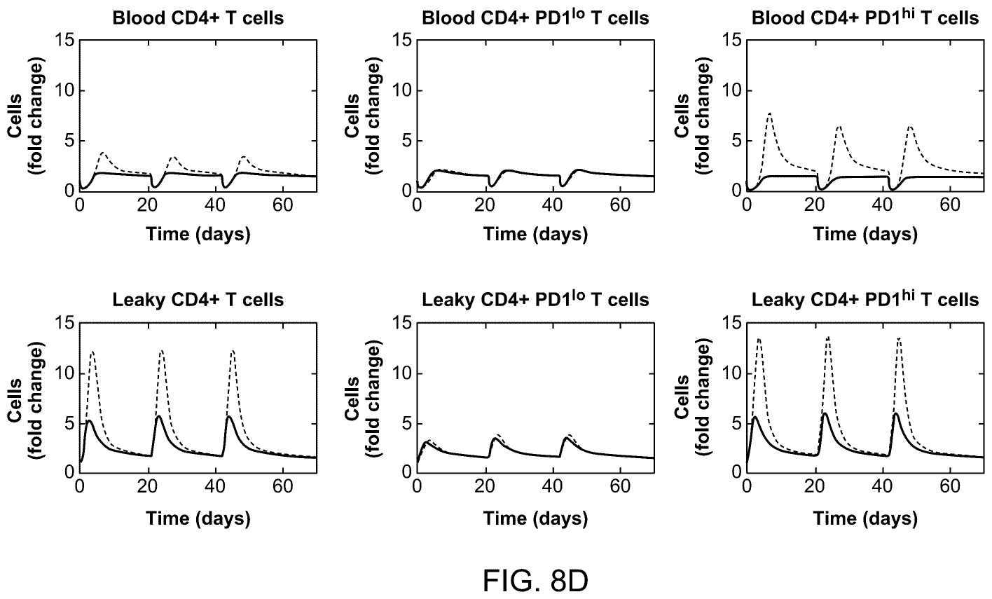

To identify how cellular expansion differs following treatment with Xmab24306 and PD1/IL-15 TaCk, the validated reference virtual cyno was used to simulate an untested treatment regimen, with molar-matched doses of Xmab24306 and PD1/IL-15 TaCk every three weeks and compared the PK & PD in both blood and leaky tissues across 70 days (see, e.g., FIGS. 8A-8D). For example, the reference virtual cyno was simulated twice to compare expansion in the central and leaky compartments following treatment with the two separate engineered IL-15 cytokines. Direct comparison of PK profiles for the same reference virtual cyno reveals greater drug exposure across time for Xmab24306 (solid line) compared to PD1/IL-15 Tack (dotted line) (see, e.g., FIG. 8A). The decline in PD1/IL-15 TaCk concentration approximately 10 days after dosing coincides with greater T cell expansion following treatment with this molecule compared to Xmab24306.

Subsequently, the question of how model-predicted cellular expansion across blood and leaky tissues differed between therapies was investigated. In blood, a similar expansion of total CD8+ T cell counts were predicted following treatment with either molecule (see, e.g., FIG. 8B), but note greater expansion of blood CD4+ T cells driven by PD1^{hi} cells following treatment with PD1/IL-15 TaCk (see, e.g., FIG. 8D). In tissue, the newly developed model predicts similar expansion dynamics as shown in blood, in that total cell expansion after PD1/IL-15 TaCk therapy is driven by PD1^{hi} cells. Additionally, the model predicts NK cell expansion in both blood and tissue following treatment with Xmab24306, but does not predict a large change for NK cells following treatment with PD1/IL-15 TaCk (see, e.g., FIG. 8C). While the cellular count in the blood is a direct consequence of previous calibration (see, e.g., FIGS. 5-6), the lack of NK cell response in the tissue constitutes a model prediction. In sum, by directly comparing the response of a single virtual subject that was treated with either Xmab24306 or PD1/IL-15 TaCk, the impact of PD1-targeting can be assessed with the latter molecule—this targeting drives the expansion of PD1^{hi} CD4+ and CD8+ T cells in tissues during treatment, a prediction which has consequences for clinical design and safety considerations.

iv. Despite Differential Binding in the Blood, Xmab24306 and PD1 IL-15 TaCk Bind to Similar Cell Populations in Tissue

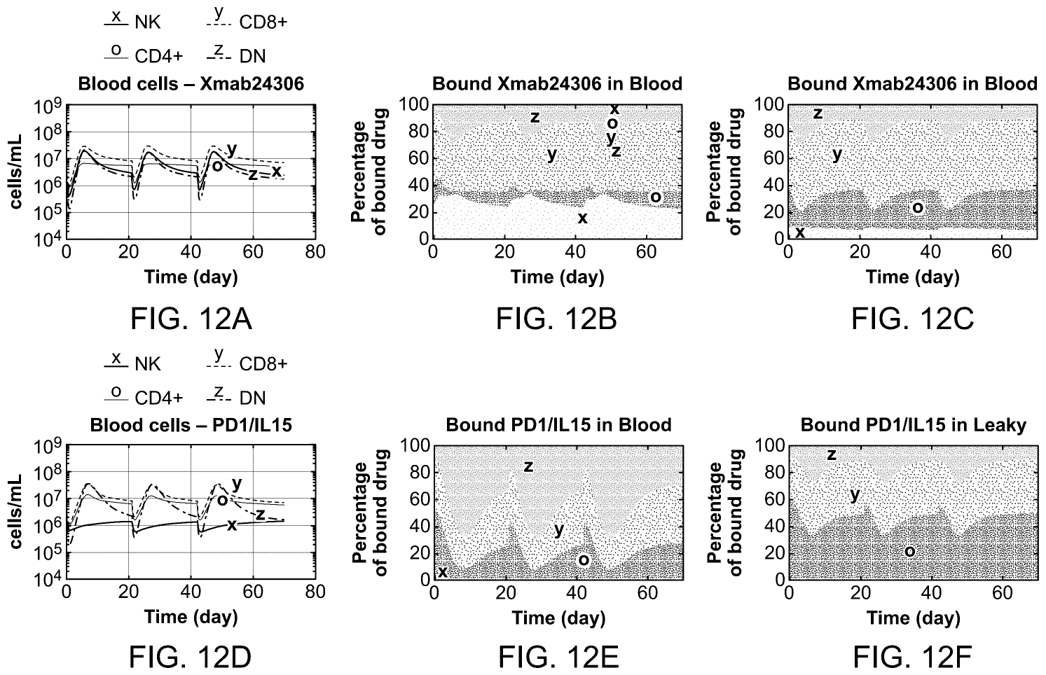

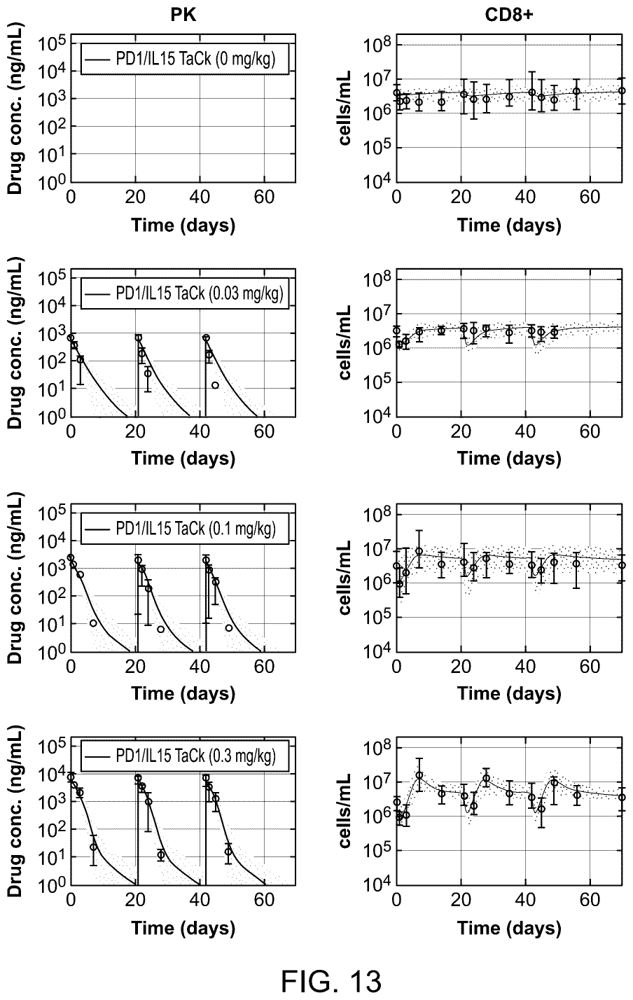

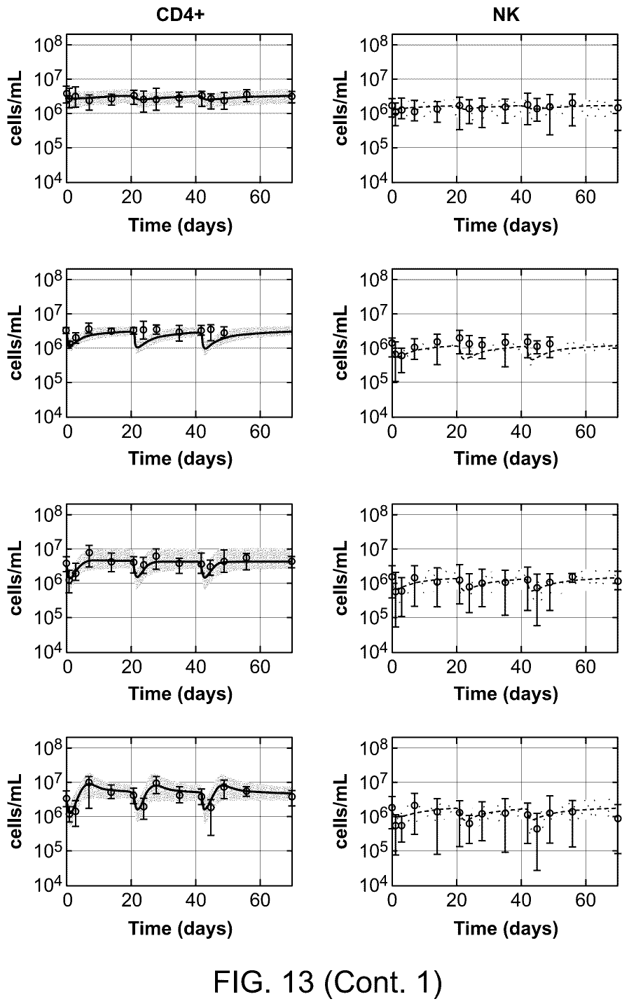

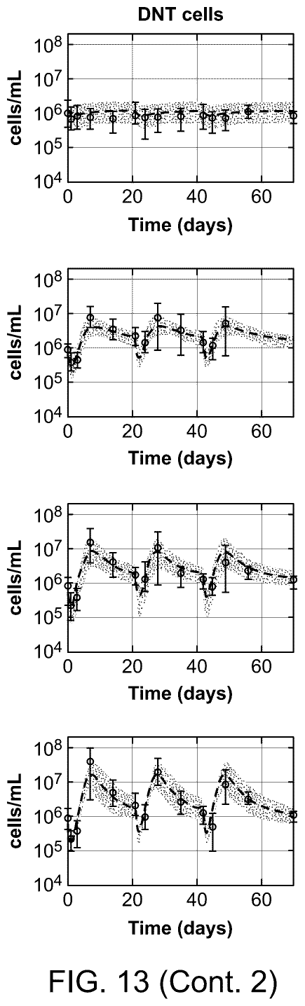

The reference virtual cyno was used to simulate Xmab24306 and PD1/IL-15 TaCk to answer two questions: 1) which cells dominate binding to Xmab24306 and PD1/IL-15 TaCk? and 2) is binding in the central compartment illustrative of binding in other tissues? FIGS. 12 and 13 illustrate total cell counts in the central compartment across time, as well as the breakdown of which cells bind to Xmab24306 (FIG. 12) and PD1/IL-15 TaCk (FIG. 13) in central and leaky compartments. Following dosing with Xmab24306, total NK cell counts and binding of drug to NK cells in the central compartment increase (see, e.g., FIG. 12), whereas NK counts in the central compartment are not altered following treatment with PD1/IL-15 TaCk (see, e.g., FIG. 13). Instead, double-negative T cells dominate drug binding and exhibit a large expansion (greater than one order of magnitude in total cell count) following treatment with PD1/IL-15 TaCk. However, within the leaky compartment, treatment with either Xmab24306 or PD1/IL-15 TaCk resulted in a similar breakdown: drug bound primarily to CD8+ T cells or CD4+ T cells. This analysis suggests a key takeaway: while cell fold-change in the central compartment may be indicative of cell fold-change in other tissues for both of these drugs (see, e.g., FIGS. 8A-8D), the central compartment may not always reflect dynamics of other measures in other tissues, particularly total cellular counts or, relatedly, which cells bind to the drug. Thus, the QSP model described herein desirably describes the dynamics in the central compartment, the tight compartment, the leaky compartment, the tumor compartment, and the lymph compartment.

v. Simulations in a Virtual Cohort Capture the Variability and Dynamics of Lymphocyte Expansion Across Multiple Dose Cohorts Following PD1 IL-15 TaCk Treatment

To address both uncertainty due to non-identifiability of parameter values as well as inter-subject variability, a virtual cyno cohort was generated to capture the observed variability within the validation preclinical PD1/IL-15 TaCk dataset. A random search method was used to generate alternate parameter values that are locally randomized around the reference virtual cyno parameter values. These parameter values were selected within reasonable parameter ranges and gQSPSim was used to guide selection of valid virtual subjects based on pre-specified acceptance criteria ranges. It was then confirmed that the virtual cohort, composed of 105 valid virtual cynos, successfully captured the variability of response for the preclinical Xmab24306 datasets. This virtual cohort could capture inherent uncertainty and variability in the predictions and was used to inform translational efforts for the PD1/IL-15 TaCk molecule.

The experimental data presented in the above Examples demonstrate that the QSP model disclosed herein was able to capture the PK and cell dynamics in the blood and predict the PK and cell expansions in the tumor and healthy tissues. This modeling framework provides a novel approach for balancing lymphocyte expansion in different tissues versus tumors based on the target expression, and could be beneficial in the early stages of drug development. This model supports preclinical, translational and early clinical development of IL-15 molecules, but could be extended to support other engineered cytokines that act to activate the immune system and expand target cells for cancer immunotherapy.

While particular alternatives of the present disclosure have been disclosed, it is to be understood that various modifications and combinations are possible and are contemplated within the true spirit and scope of the appended claims. There is no intention, therefore, of limitations to the exact abstract and disclosure herein presented.

1. A method, comprising:
determining, by at least one data processor and after delivery of a cytokine molecule, (a) a dynamics parameter corresponding to a plurality of target lymphocytes within a central compartment of a patient and (b) a binding parameter between the cytokine molecule and the plurality of target lymphocytes within the central compartment, wherein the plurality of target lymphocytes include a CD8+ T cell, a CD4+ T cell, a CD4−/CD8− (Double Negative) T cell, and a natural killer (NK) cell;
determining, by the at least one data processor, (a) a first target cell trafficking rate of the plurality of target lymphocytes between the central compartment and a tight compartment of the patient and (b) a first cytokine partitioning rate of the cytokine molecule between the central compartment and the tight compartment;
determining, by the at least one data processor, (a) the dynamics parameter corresponding to the plurality of target lymphocytes within the tight compartment and (b) the binding parameter between the cytokine molecule and the plurality of target lymphocytes within the tight compartment;
determining, by the at least one data processor, (a) a second target cell trafficking rate of the plurality of target lymphocytes between the central compartment and a leaky compartment of the patient and (b) a second cytokine partitioning rate of the cytokine molecule between the central compartment and the leaky compartment;
determining, by the at least one data processor, (a) the dynamics parameter corresponding to the plurality of target lymphocytes within the leaky compartment and (b) the binding parameter between the cytokine molecule and the plurality of target lymphocytes within the leaky compartment;
determining, by the at least one data processor, (a) a third target cell trafficking rate of the plurality of target lymphocytes between the central compartment and a tumor compartment of the patient and (b) a third cytokine partitioning rate of the cytokine molecule between the central compartment and the tumor compartment;
determining, by the at least one data processor, (a) the dynamics parameter corresponding to the plurality of target lymphocytes within the tumor compartment and (b) the binding parameter between the cytokine molecule and the plurality of target lymphocytes within the tumor compartment;
determining, by the at least one data processor, a fourth cytokine partitioning rate of the cytokine molecule from each of the tight compartment, the leaky compartment, and the tumor compartment, to a lymph compartment of the patient; and
determining, by the at least one data processor and based on the dynamics parameter, the binding parameter, the first target cell trafficking rate, the second target cell trafficking rate, the third target cell trafficking rate, the first cytokine partitioning rate, the second cytokine partitioning rate, the third cytokine partitioning rate, and the fourth cytokine partitioning rate, a distribution of each of the plurality of target lymphocytes in the central compartment, the tight compartment, the leaky compartment, and the tumor compartment, over time.

2. The method of claim 1, wherein the distribution includes: a first distribution corresponding to the cytokine molecule delivered to the patient at a first time point; and a second distribution corresponding to the cytokine molecule delivered to the patient at a second time point; and wherein the method comprises: determining, by the at least one data processor and based at least on the first distribution and the second distribution, a response of a second patient to a cancer immunotherapy including the cytokine molecule.

3. The method of claim 1, wherein each of the plurality of target lymphocytes includes a first subpopulation associated with high PD1 expression and a second subpopulation associated with low PD1 expression; and wherein the method further comprises: determining the dynamics parameter for the first subpopulation and the second subpopulation for each of the plurality of target lymphocytes in the central compartment, the tight compartment, the leaky compartment, and the tumor compartment.

4. The method of claim 1, wherein the dynamics parameter includes at least one of a quantity, a proliferation, an expansion, a contraction, a persistence, and a differentiation of the plurality of target lymphocytes within the central compartment, the tight compartment, the leaky compartment, and the tumor compartment.

5. The method of claim 1, wherein the binding parameter includes at least one of an affinity, an avidity, a concentration of free receptors at a surface of each of the plurality of target lymphocytes within the central compartment, the tight compartment, the leaky compartment, and the tumor compartment, a concentration of single bound drug: receptor complexes at the surface of each of the plurality of target lymphocytes within the central compartment, the tight compartment, the leaky compartment, and the tumor compartment, and a concentration fully bound cytokine molecule complexes at the surface of each of the plurality of target lymphocytes within the central compartment, the tight compartment, the leaky compartment, and the tumor compartment.

6. (canceled)

7. The method of claim 1, wherein the binding parameter is associated with expression of at least one of an interleukin 15 (IL-15) receptor and a PD1 receptor on a surface of each of the plurality of target lymphocytes within the central compartment, the tight compartment, the leaky compartment, and the tumor compartment.

8. (canceled)

9. The method of claim 1, wherein the cytokine molecule includes interleukin 2 (IL-2), interleukin 7 (IL-7), interleukin 12 (IL-12), interleukin 15 (IL-15), interleukin 21 (IL-21), interferon, Xmab24306, PD1/IL15 TaCk, or RSV IL-15 TaCk.

10-19. (canceled)

20. A system, comprising:
at least one data processor; and
at least one memory storing instructions, which when executed by the at least one data processor, result in operations comprising:
determining, by at least one data processor and after delivery of a cytokine molecule, (a) a dynamics parameter corresponding to a plurality of target lymphocytes within a central compartment of a patient and (b) a binding parameter between the cytokine molecule and the plurality of target lymphocytes within the central compartment, wherein the plurality of target lymphocytes include a CD8+ T cell, a CD4+ T cell, a CD4−/CD8− (Double Negative) T cell, and a natural killer (NK) cell;
determining, by the at least one data processor, (a) a first target cell trafficking rate of the plurality of target lymphocytes between the central compartment and a tight compartment of the patient and (b) a first cytokine partitioning rate of the cytokine molecule between the central compartment and the tight compartment;
determining, by the at least one data processor, (a) the dynamics parameter corresponding to the plurality of target lymphocytes within the tight compartment and (b) the binding parameter between the cytokine molecule and the plurality of target lymphocytes within the tight compartment;
determining, by the at least one data processor, (a) a second target cell trafficking rate of the plurality of target lymphocytes between the central compartment and a leaky compartment of the patient and (b) a second cytokine partitioning rate of the cytokine molecule between the central compartment and the leaky compartment;
determining, by the at least one data processor, (a) the dynamics parameter corresponding to the plurality of target lymphocytes within the leaky compartment and (b) the binding parameter between the cytokine molecule and the plurality of target lymphocytes within the leaky compartment;
determining, by the at least one data processor, (a) a third target cell trafficking rate of the plurality of target lymphocytes between the central compartment and a tumor compartment of the patient and (b) a third cytokine partitioning rate of the cytokine molecule between the central compartment and the tumor compartment;
determining, by the at least one data processor, (a) the dynamics parameter corresponding to the plurality of target lymphocytes within the tumor compartment and (b) the binding parameter between the cytokine molecule and the plurality of target lymphocytes within the tumor compartment;
determining, by the at least one data processor, a fourth cytokine partitioning rate of the cytokine molecule from each of the tight compartment, the leaky compartment, and the tumor compartment, to a lymph compartment of the patient; and
determining, by the at least one data processor and based on the dynamics parameter, the binding parameter, the first target cell trafficking rate, the second target cell trafficking rate, the third target cell trafficking rate, the first cytokine partitioning rate, the second cytokine partitioning rate, the third cytokine partitioning rate, and the fourth cytokine partitioning rate, a distribution of each of the plurality of target lymphocytes in the central compartment, the tight compartment, the leaky compartment, and the tumor compartment, over time.

21. The system of claim 20, wherein the distribution includes: a first distribution corresponding to the cytokine molecule delivered to the patient at a first time point; and a second distribution corresponding to the cytokine molecule delivered to the patient at a second time point; and wherein the operations further comprise: determining, by the at least one data processor and based at least on the first distribution and the second distribution, a response of a second patient to a cancer immunotherapy including the cytokine molecule.

22. The system of claim 20, wherein each of the plurality of target lymphocytes includes a first subpopulation associated with high PD1 expression and a second subpopulation associated with low PD1 expression; and wherein the operations further comprise: determining the dynamics parameter for the first subpopulation and the second subpopulation for each of the plurality of target lymphocytes in the central compartment, the tight compartment, the leaky compartment, and the tumor compartment.

23. The system of claim 20, wherein the dynamics parameter includes at least one of a quantity, a proliferation, an expansion, a contraction, a persistence, and a differentiation of the plurality of target lymphocytes within the central compartment, the tight compartment, the leaky compartment, and the tumor compartment.

24. The system of claim 20, wherein the binding parameter includes at least one of an affinity, an avidity, a concentration of free receptors at a surface of each of the plurality of target lymphocytes within the central compartment, the tight compartment, the leaky compartment, and the tumor compartment, a concentration of single bound drug: receptor complexes at the surface of each of the plurality of target lymphocytes within the central compartment, the tight compartment, the leaky compartment, and the tumor compartment, and a concentration fully bound cytokine molecule complexes at the surface of each of the plurality of target lymphocytes within the central compartment, the tight compartment, the leaky compartment, and the tumor compartment.

25. (canceled)

26. The system of claim 20, wherein the binding parameter is associated with expression of at least one of an interleukin 15 (IL-15) receptor and a PD1 receptor on a surface of each of the plurality of target lymphocytes within the central compartment, the tight compartment, the leaky compartment, and the tumor compartment.

27. (canceled)

28. The system of claim 20, wherein the cytokine molecule includes interleukin 2 (IL-2), interleukin 7 (IL-7), interleukin 12 (IL-12), interleukin 15 (IL-15), interleukin 21 (IL-21), interferon, Xmab24306, PD1/IL15 TaCk, or RSV IL-15 TaCk.

29-38. (canceled)

39. A non-transitory computer-readable medium storing instructions, which
when executed by at least one data processor, result in operations comprising:
determining, by at least one data processor and after delivery of a cytokine molecule, (a) a dynamics parameter corresponding to a plurality of target lymphocytes within a central compartment of a patient and (b) a binding parameter between the cytokine molecule and the plurality of target lymphocytes within the central compartment, wherein the plurality of target lymphocytes include a CD8+ T cell, a CD4+ T cell, a CD4−/CD8− (Double Negative) T cell, and a natural killer (NK) cell;
determining, by the at least one data processor, (a) a first target cell trafficking rate of the plurality of target lymphocytes between the central compartment and a tight compartment of the patient and (b) a first cytokine partitioning rate of the cytokine molecule between the central compartment and the tight compartment;
determining, by the at least one data processor, (a) the dynamics parameter corresponding to the plurality of target lymphocytes within the tight compartment and (b) the binding parameter between the cytokine molecule and the plurality of target lymphocytes within the tight compartment;
determining, by the at least one data processor, (a) a second target cell trafficking rate of the plurality of target lymphocytes between the central compartment and a leaky compartment of the patient and (b) a second cytokine partitioning rate of the cytokine molecule between the central compartment and the leaky compartment;
determining, by the at least one data processor, (a) the dynamics parameter corresponding to the plurality of target lymphocytes within the leaky compartment and (b) the binding parameter between the cytokine molecule and the plurality of target lymphocytes within the leaky compartment;
determining, by the at least one data processor, (a) a third target cell trafficking rate of the plurality of target lymphocytes between the central compartment and a tumor compartment of the patient and (b) a third cytokine partitioning rate of the cytokine molecule between the central compartment and the tumor compartment;
determining, by the at least one data processor, (a) the dynamics parameter corresponding to the plurality of target lymphocytes within the tumor compartment and (b) the binding parameter between the cytokine molecule and the plurality of target lymphocytes within the tumor compartment;
determining, by the at least one data processor, a fourth cytokine partitioning rate of the cytokine molecule from each of the tight compartment, the leaky compartment, and the tumor compartment, to a lymph compartment of the patient; and
determining, by the at least one data processor and based on the dynamics parameter, the binding parameter, the first target cell trafficking rate, the second target cell trafficking rate, the third target cell trafficking rate, the first cytokine partitioning rate, the second cytokine partitioning rate, the third cytokine partitioning rate, and the fourth cytokine partitioning rate, a distribution of each of the plurality of target lymphocytes in the central compartment, the tight compartment, the leaky compartment, and the tumor compartment, over time.

40. The non-transitory computer-readable medium of claim 39, wherein the distribution includes: a first distribution corresponding to the cytokine molecule delivered to the patient at a first time point; and a second distribution corresponding to the cytokine molecule delivered to the patient at a second time point; and wherein the operations further comprise: determining, by the at least one data processor and based at least on the first distribution and the second distribution, a response of a second patient to a cancer immunotherapy including the cytokine molecule.

41. The non-transitory computer-readable medium of claim 39, wherein each of the plurality of target lymphocytes includes a first subpopulation associated with high PD1 expression and a second subpopulation associated with low PD1 expression; and wherein the operations further comprise: determining the dynamics parameter for the first subpopulation and the second subpopulation for each of the plurality of target lymphocytes in the central compartment, the tight compartment, the leaky compartment, and the tumor compartment.

42. The non-transitory computer-readable medium of claim 39, wherein the dynamics parameter includes at least one of a quantity, a proliferation, an expansion, a contraction, a persistence, and a differentiation of the plurality of target lymphocytes within the central compartment, the tight compartment, the leaky compartment, and the tumor compartment.

43. The non-transitory computer-readable medium of claim 39, wherein the binding parameter includes at least one of an affinity, an avidity, a concentration of free receptors at a surface of each of the plurality of target lymphocytes within the central compartment, the tight compartment, the leaky compartment, and the tumor compartment, a concentration of single bound drug: receptor complexes at the surface of each of the plurality of target lymphocytes within the central compartment, the tight compartment, the leaky compartment, and the tumor compartment, and a concentration fully bound cytokine molecule complexes at the surface of each of the plurality of target lymphocytes within the central compartment, the tight compartment, the leaky compartment, and the tumor compartment.

44. (canceled)

45. The non-transitory computer-readable medium of claim 39, wherein the binding parameter is associated with expression of at least one of an interleukin 15 (IL-15) receptor and a PD1 receptor on a surface of each of the plurality of target lymphocytes within the central compartment, the tight compartment, the leaky compartment, and the tumor compartment.

46. (canceled)

47. The non-transitory computer-readable medium of claim 39, wherein the cytokine molecule includes interleukin 2 (IL-2), interleukin 7 (IL-7), interleukin 12 (IL-12), interleukin 15 (IL-15), interleukin 21 (IL-21), interferon, Xmab24306, PD1/IL15 TaCk, or RSV IL-15 TaCk.

48-57. (canceled)
